SEQUENCE LISTING (72 sequences)

SEQ ID NO: 1 - DNA, 20 bp, Artificial
  Synthetic DNA construct for 5′ end primer for mouse Nanog
gcaagcggtggcagaaaaac

SEQ ID NO: 2 - DNA, 22 bp, Artificial
  Synthetic DNA construct for 3′ end primer for mouse Nanog
gcaatggatgctgggatactcc

SEQ ID NO: 3 - DNA, 26 bp, Artificial
  Synthetic DNA construct for 5′ end primer for mouse Klf4
atctcaaggcacacctgcgaactcac

SEQ ID NO: 4 - DNA, 22 bp, Artificial
  Synthetic DNA construct for 3′ end primer for mouse Klf4
cacacttctggcactgaaaggg

SEQ ID NO: 5 - DNA, 22 bp, Artificial
  Synthetic DNA construct for 5′ end primer for mouse Lin28
gaacatgcagaagcgaagatcc

SEQ ID NO: 6 - DNA, 22 bp, Artificial
  Synthetic DNA construct for 3′ end primer for mouse Lin28
gttgatgctttggcaaaagtgg

SEQ ID NO: 7 - DNA, 20 bp, Artificial
  Synthetic DNA construct for 5′ end primer for mouse Cct4
gttggagaaggtggaaccaa

SEQ ID NO: 8 - DNA, 19 bp, Artificial
  Synthetic DNA construct for 3′ end primer for mouse Cct4
ctccttctgcagggctttc

SEQ ID NO: 9 - DNA, 18 bp, Artificial
  Synthetic DNA construct for 5′ end primer for mouse Sox2
acagctacgcgcacatga

SEQ ID NO: 10 - DNA, 18 bp, Artificial
  Synthetic DNA construct for 3′ end primer for mouse Sox2
ggtagcccagctgctcct

SEQ ID NO: 11 - DNA, 20 bp, Artificial
  Synthetic DNA construct for 5′ end primer for mouse cMyc
cctagtgctgcatgaggaga

SEQ ID NO: 12 - DNA, 22 bp, Artificial
  Synthetic DNA construct for 3′ end primer for mouse cMyc
tccacagacaccacatcaattt

SEQ ID NO: 13 - DNA, 19 bp, Artificial
  Synthetic DNA construct for 5′ end primer for mouse FGF2
cggctctactgcaagaacg

SEQ ID NO: 14 - DNA, 22 bp, Artificial
  Synthetic DNA construct for 3′ end primer for mouse FGF2
tgcttggagttgtagtttgacg

SEQ ID NO: 15 - DNA, 20 bp, Artificial
  Synthetic DNA construct for 5′ end primer for mouse TGF-beta-1
tggagcaacatgtggaactc

SEQ ID NO: 16 - DNA, 18 bp, Artificial
  Synthetic DNA construct for 3′ end primer for mouse TGF-beta-1
cagcagccggttaccaag

SEQ ID NO: 17 - DNA, 20 bp, Artificial
  Synthetic DNA construct for 5′ end primer for mouse fibronectin
cggagagagtgcccctacta

SEQ ID NO: 18 - DNA, 20 bp, Artificial
  Synthetic DNA construct for 3′ end primer for mouse fibronectin
cgatattggtgaatcgcaga

SEQ ID NO: 19 - DNA, 19 bp, Artificial
  Synthetic DNA construct for 5′ end primer for mouse vitronectin
ctggggcagatcctctgat

SEQ ID NO: 20 - DNA, 19 bp, Artificial
  Synthetic DNA construct for 3′ end primer for mouse vitronectin
actttccctggcacaccat

SEQ ID NO: 21 - DNA, 20 bp, Artificial
  Synthetic DNA construct for 5′ end primer for mouse Activin A
atcatcacctttgccgagtc

SEQ ID NO: 22 - DNA, 20 bp, Artificial
  Synthetic DNA construct for 3′ end primer for mouse Activin A
acaggtcactgccttccttg

SEQ ID NO: 23 - DNA, 20 bp, Artificial
  Synthetic DNA construct for 5′ end primer for mouse LIF
cccagggatttccaggtact

SEQ ID NO: 24 - DNA, 21 bp, Artificial
  Synthetic DNA construct for 3′ end primer for mouse LIF
tcaactttgccagattccatc

SEQ ID NO: 25 - DNA, 20 bp, Artificial
  Synthetic DNA construct for 5′ end primer for human Nanog
ccaacatcctgaacctcagc

SEQ ID NO: 26 - DNA, 19 bp, Artificial
  Synthetic DNA construct for 3′ end primer for human Nanog
gctattcttcggccagttg

SEQ ID NO: 27 - DNA, 19 bp, Artificial
  Synthetic DNA construct for 5′ end primer for human Klf4
gggagaagacactgcgtca

SEQ ID NO: 28 - DNA, 18 bp, Artificial
  Synthetic DNA construct for 3′ end primer for human Klf4
ggaagcactgggggaagt

SEQ ID NO: 29 - DNA, 20 bp, Artificial
  Synthetic DNA construct for 5′ end primer for human Lin28
gaagcgcagatcaaaaggag

SEQ ID NO: 30 - DNA, 20 bp, Artificial
  Synthetic DNA construct for 3′ end primer for human Lin28
gctgatgctctggcagaagt

SEQ ID NO: 31 - DNA, 18 bp, Artificial
  Synthetic DNA construct for 5′ end primer for human Oct4
gcaaaacccggaggagtc

SEQ ID NO: 32 - DNA, 21 bp, Artificial
  Synthetic DNA construct for 3′ end primer for human Oct4
ccacatcggcctgtgtatatc

SEQ ID NO: 33 - DNA, 23 bp, Artificial
  Synthetic DNA construct for 5′ end primer for human Sox2
ttgctgcctctttaagactagga

SEQ ID NO: 34 - DNA, 20 bp, Artificial
  Synthetic DNA construct for 3′ end primer for human Sox2
ctggggctcaaacttctctc

SEQ ID NO: 35 - DNA, 18 bp, Artificial
  Synthetic DNA construct for 5′ end primer human cMyc
caccagcagcgactctga

SEQ ID NO: 36 - DNA, 22 bp, Artificial
  Synthetic DNA construct for 3′ end primer human cMyc
gatccagactctgaccttttgc

SEQ ID NO: 37 - DNA, 21 bp, Artificial
  Synthetic DNA construct for 5′ end primer for human Zfp42
gcgtcataaggggtgagtttt

SEQ ID NO: 38 - DNA, 21 bp, Artificial
  Synthetic DNA construct for 3′ end primer for human Zfp42
agaacattcaagggagcttgc

SEQ ID NO: 39 - DNA, 21 bp, Artificial
  Synthetic DNA construct for 5′ end primer for human Dppa2
tggtgtcaacaactcggtttg

SEQ ID NO: 40 - DNA, 22 bp, Artificial
  Synthetic DNA construct for 3′ end primer for human Dppa2
ctcgaacatcgctgtaatctgg

SEQ ID NO: 41 - DNA, 20 bp, Artificial
  Synthetic DNA construct for 5′ end primer for human GATA2
aaggctcgttcctgttcaga

SEQ ID NO: 42 - DNA, 19 bp, Artificial
  Synthetic DNA construct for 53 end primer for human GATA2
ggcattgcacaggtagtgg

SEQ ID NO: 43 - DNA, 20 bp, Artificial
  Synthetic DNA construct for 5′ end primer for human Nestin
tgcgggctactgaaaagttc

SEQ ID NO: 44 - DNA, 20 bp, Artificial
  Synthetic DNA construct for 3′ end primer for human Nestin
tgtaggccctgtttctcctg

SEQ ID NO: 45 - DNA, 20 bp, Artificial
  Synthetic DNA construct for 5′ end primer for human SMA
ctgttccagccatccttcat

SEQ ID NO: 46 - DNA, 22 bp, Artificial
  Synthetic DNA construct for 3′ end primer for human SMA
tcatgatgctgttgtaggtggt

SEQ ID NO: 47 - DNA, 21 bp, Artificial
  Synthetic DNA construct for 5′ end primer for human Actinin
gacgtggcagagaagtacctg

SEQ ID NO: 48 - DNA, 20 bp, Artificial
  Synthetic DNA construct for 3′ end primer for human Actinin
ggcagttccaacgatgtctt

SEQ ID NO: 49 - DNA, 23 bp, Artificial
  Synthetic DNA construct for 5′ end primer for human AFP
aagaatttcagcatgattttcca

SEQ ID NO: 50 - DNA, 20 bp, Artificial
  Synthetic DNA construct for 3′ end primer for human AFP
cacccacttcatggttgcta

SEQ ID NO: 51 - DNA, 20 bp, Artificial
  Synthetic DNA construct for 5′ end primer for human Sox7
gagcagtgtggacacgtacc

SEQ ID NO: 52 - DNA, 20 bp, Artificial
  Synthetic DNA construct for 3′ end primer for human Sox7
gtccaggggagacatttcag

SEQ ID NO: 53 - DNA, 18 bp, Artificial
  Synthetic DNA construct for 5′ end primer for human PDX1
aagctcacgcgtggaaag

SEQ ID NO: 54 - DNA, 22 bp, Artificial
  Synthetic DNA construct for 53 end primer for human PDX1
gccgtgagatgtacttgttgaa

SEQ ID NO: 55 - DNA, 18 bp, Artificial
  Synthetic DNA construct for 5′ end primer for human FGF2
ttcttcctgcgcatccac

SEQ ID NO: 56 - DNA, 23 bp, Artificial
  Synthetic DNA construct for 3′ end primer for human FGF2
tgcttgaagttgtagcttgatgt

SEQ ID NO: 57 - DNA, 18 bp, Artificial
  Synthetic DNA construct for 5′ end primer for human TGF-beta-1
gcagcacgtggagctgta

SEQ ID NO: 58 - DNA, 18 bp, Artificial
  Synthetic DNA construct for 3′ end primer for human TGF-beta-1
cagccggttgctgaggta

SEQ ID NO: 59 - DNA, 22 bp, Artificial
  Synthetic DNA construct for 5′ end primer for human fibronectin
ctggccgaaaatacattgtaaa

SEQ ID NO: 60 - DNA, 18 bp, Artificial
  Synthetic DNA construct for 3′ end primer for human fibronectin
ccacagtcgggtcaggag

SEQ ID NO: 61 - DNA, 18 bp, Artificial
  Synthetic DNA construct for 5′ end primer for human vitronectin
tgagtgcaagccccaagt

SEQ ID NO: 62 - DNA, 20 bp, Artificial
  Synthetic DNA construct for 3′ end primer for human vitronectin
gccatcgtcatagaccgtgt

SEQ ID NO: 63 - DNA, 20 bp, Artificial
  Synthetic DNA construct for 5′ end primer for human activin A
ctcggagatcatcacgtttg

SEQ ID NO: 64 - DNA, 20 bp, Artificial
  Synthetic DNA construct for 3′ end primer for human activin A
ccttggaaatctcgaagtgc

SEQ ID NO: 65 - DNA, 23 bp, Artificial
  Synthetic DNA construct for 5′ end primer for LTR of retrovirus construct
ggaatgaaagaccccacctgtag

SEQ ID NO: 66 - DNA, 24 bp, Artificial
  Synthetic DNA construct for 3′ end primer for LTR of retrovirus construct
gcgagaagcgaactgattggttag

SEQ ID NO: 67 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 68 - protein, 265 aa, Homo sapiens
MHFYRLFLGATRRFLNPEWKGEIDNWCVYVLTSLLPFKIQSQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 69 - protein, 190 aa, Homo sapiens
MGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 70 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 71 - protein, 479 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 72 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

(19) United States
(12) Patent Application Publication
(10) Pub. No.: US 2012/0263689 A1
(43) Pub. Date: Oct. 18, 2012

(54) ADIPOSE-DERIVED INDUCED PLURIPOTENT STEM CELLS

(75) Inventors: Shigeki Sugii, San Diego, CA (US); Ronald M. Evans, La Jolla, CA (US); Juan Carlos Izpisua Belmonte, La Jolla, CA (US); Yasuyuki Kida, San Diego, CA (US)

(73) Assignee: The Salk Institute for Biological Studies, La Jolla, CA (US)

(21) Appl. No.: 13/395,093

(22) PCT Filed: Sep. 10, 2010
(86) PCT No.: PCT/US10/48507

§ 371 Date: Jun. 25, 2012

Related U.S. Application Data
(60) Provisional application No. 61/241,122, filed on Sep. 10, 2009.
(60) Provisional application No. 61/298,164, filed on Jan. 25, 2010.

(51) Int. Cl. C12N 5/0775; A61K 35/12; A61P 17/02; C12N 15/87

(52) U.S. Cl. 424/93.21; 435/465; 435/325; 435/375; 424/93.7

(57) ABSTRACT

Methods and compositions for generating adipose-derived induced pluripotent stem cells for humans and animals and their use are provided.

### CROSS-REFERENCES TO RELATED APPLICATIONS

This application claims the benefit of U.S. Provisional Application No. 61/241,122, filed Sep. 10, 2009, and U.S. Provisional Application No. 61/298,164, filed Jan. 25, 2010, the contents or which are incorporated herein for all purposes in their entirety.

### STATEMENT AS TO RIGHTS TO INVENTIONS MADE UNDER FEDERALLY SPONSORED RESEARCH OR DEVELOPMENT

The present invention was supported by the following grants from the US National Institutes of Health: HD027183, DK057978 and DK062434. The United States Government has certain rights in the invention.

### BACKGROUND OF THE INVENTION

In the presence of feeder cells induced pluripotent stem (iPS) cells have been successfully generated from fibroblasts, peripheral blood cells, neural stem cells and keratinocytes (12, 17, 18). Co-culture of iPS and ES cells with supporting cell layers such as mouse embryonic fibroblasts is generally used to maintain their proliferation and self-renewal under a pluripotent state. However, contamination with feeder cells, animal products and xenobiotics remain a serious concern for maintaining functional integrity of both ES and iPS cells. For example, in one study, co-culturing of human Es cells with mouse feeders or animal serum led to the expression of a nonhuman cell surface antigen (sialic acid Neu5Gc) that should be immunogenic when transplanted (7). In addition, the presence of unknown animal sources in the media makes it more tedious to perform quality control and runs a risk of animal-derived pathogen transmission. Thus in order to clinically translate iPS technology into therapies, it is very important to establish a GMP-compliant system to produce and maintain iPS cells. Achieving feeder- and xeno-free conditions to grow these cells is one key step toward such a system. The present invention provides compositions and methods for the generation of iPS cells from readily accessible tissues and further overcomes these and other problems in the art. Provided herein are compositions and methods to prepare iPS cells from adipose-derived cells with high efficiency in the absence of feeder cells. Adipose-derived iPS as described herein represent useful tools in regenerative therapeutics, including the establishment of an iPS library derived from readily obtainable human fat biopsies that cover comprehensive HLA haplotypes. The ability to generate induced pluripotent stem cells at high efficiency from an abundant source of progenitor cell populations under feeder-free conditions represents a highly desirable tool for a wide range of therapeutic approaches. Further, in the methods provided herein adipose-derived stem cells may be used as abundant source of patient-specific feeder cells. Adipose-derived stem cells may be used as feeder cells during the reprogramming process of primary cells such as fibroblasts, peripheral blood cells, neural stem cells and keratinocytes.

### SUMMARY OF THE INVENTION

Provided herein are, inter alia, highly efficient methods and compositions for making and using adipose-derived induced pluripotent stem cells. In some embodiments, the methods and compositions may be used in personalized medicine applications. For example, an induced pluripotent stem cell may be derived from a patient's adipose tissue (an adipose-derived induced pluripotent stem cell) and used in methods to treat the patient or used in methods to produce compositions useful in treating the patient (e.g. xeno-free liquid composition or xeno-free growth media). The personalized medicine approach may be used to address problems related to treatment composition contamination and rejection of transplanted materials.

In one aspect, a method for preparing an adipose-derived induced pluripotent stem cell is provided. The method includes transfecting an adipose-derived stem cell with a nucleic acid encoding a cMYC protein, a nucleic acid encoding an OCT4 protein, a nucleic acid encoding a KLF4 protein and a nucleic acid encoding a SOX2 protein to form a transfected adipose-derived stem cell. The transfected adipose-derived stem cell is allowed to divide thereby forming the adipose-derived induced pluripotent stem cell.

In another aspect, a method for preparing an adipose-derived induced pluripotent stem cell is provided. The method includes transfecting an adipose-derived stem cell with a nucleic acid encoding an OCT4 protein and a nucleic acid encoding a SOX2 protein to form a transfected adipose-derived stem cell. The transfected adipose-derived stem cell is allowed to divide thereby forming the adipose-derived induced pluripotent stem cell.

In another aspect, an adipose-derived stem cell including a nucleic acid encoding a cMYC protein, a nucleic acid encoding an OCT4 protein, a nucleic acid encoding a KLF4 protein and a nucleic acid encoding a SOX2 protein is provided.

In another aspect, an adipose-derived stem cell including a nucleic acid encoding an OCT4 protein and a nucleic acid encoding a SOX2 protein is provided.

In another aspect, a method for producing a somatic cell is provided. The method includes contacting an adipose-derived induced pluripotent stem cell with cellular growth factors. The adipose-derived induced pluripotent stem cell is allowed to divide, thereby forming the somatic cell.

In another aspect, a method of treating a mammal in need of tissue repair is provided. The method includes administering an adipose-derived induced pluripotent stem to the mammal. The adipose-derived induced pluripotent stem cell is allowed to divide and differentiate into somatic cells in the mammal, thereby providing tissue repair in the mammal.

In another aspect, a method of culturing an induced pluripotent stem cell is provided. The method includes contacting the induced pluripotent stem cell with a growth medium. The growth medium includes an adipose-derived induced pluripotent stem cell. The method further comprises allowing the induced pluripotent stem cell to divide in the presence of the growth medium.

In another aspect, a xeno-free liquid composition is provided. The xeno-free liquid composition is prepared by a process comprising transfecting an adipose-derived stem cell with a nucleic acid encoding a cMYC protein, a nucleic acid encoding an OCT4 protein, a nucleic acid encoding a KLF4 protein and a nucleic acid encoding a SOX2 protein to form a transfected adipose-derived stem cell. The transfected adipose-derived stem cell is allowed to divide in a xeno-free liquid. The xeno-free liquid is isolated thereby preparing the xeno-free liquid composition.

In another aspect, a method of culturing an induced pluripotent stem cell is provided. The method includes contacting the induced pluripotent stem cell with a xeno-free growth medium. The xeno-free growth medium includes the xeno-free liquid composition prepared by the methods provided herein. The induced pluripotent stem cell is allowed to divide in the presence of the growth media.

### BRIEF DESCRIPTION OF THE DRAWINGS

FIG. 1: Generation of ES-like iPS cell lines from mouse adipose-derived cells. (FIG. 1A) SVF Lin⁻ and Lin⁺ cells transduced with four reprogramming factors are stained for Nanog by immunohistochemistry after 7 days. Both whole-plate (left) and two representative images of individual colonies (right) are shown. Note that almost all Lin⁺ derived colonies are negative for Nanog. (FIG. 1B) mADS-derived iPS clones after derivation. (FIG. 1C) Histograms depict gene expression analysis by qPCR which indicates that two representative mADS-derived iPS clones express comparable pluripotent markers to mouse ES cells, in contrast to somatic cells, MEFs, mADS and SVF cells. Order (left to right): ES, IPS#1, IPS#2, MEF, mADS and SVF.

FIG. 2: In vivo pluripotency tests of mouse adipose-derived iPS cell lines. (FIGS. 2A-2C) H&E staining of teratomas developed from mADS-derived iPS cells shows their contribution to ectoderm (B: brain, SE: squamous epithelium), mesoderm (Ct: cartilage, SM: smooth muscle, Ad: adipose), and endoderm (GC: goblet cells, P: pancreas). (FIG. 2D) A representative picture of a chimera mouse (right) produced from mADS-derived iPS cells. (FIG. 2E) Genotyping PCR analysis demonstrates the presence of retrovirus-specific DNA elements (LTR; long terminal repeat) in 50% of the offspring (#2, 4, 6, 7, 9) produced by crossing the chimera with wild-type mice. Endogenous GAPDH gene products are used as an internal control.

FIG. 3: Reprogramming of human adipose-derived cells. (FIG. 3A)

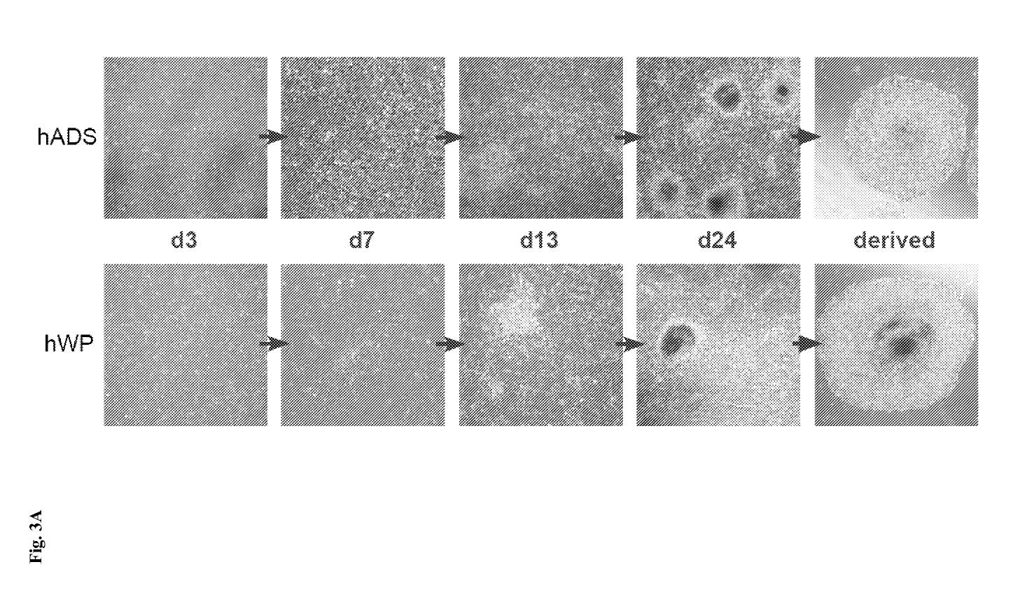

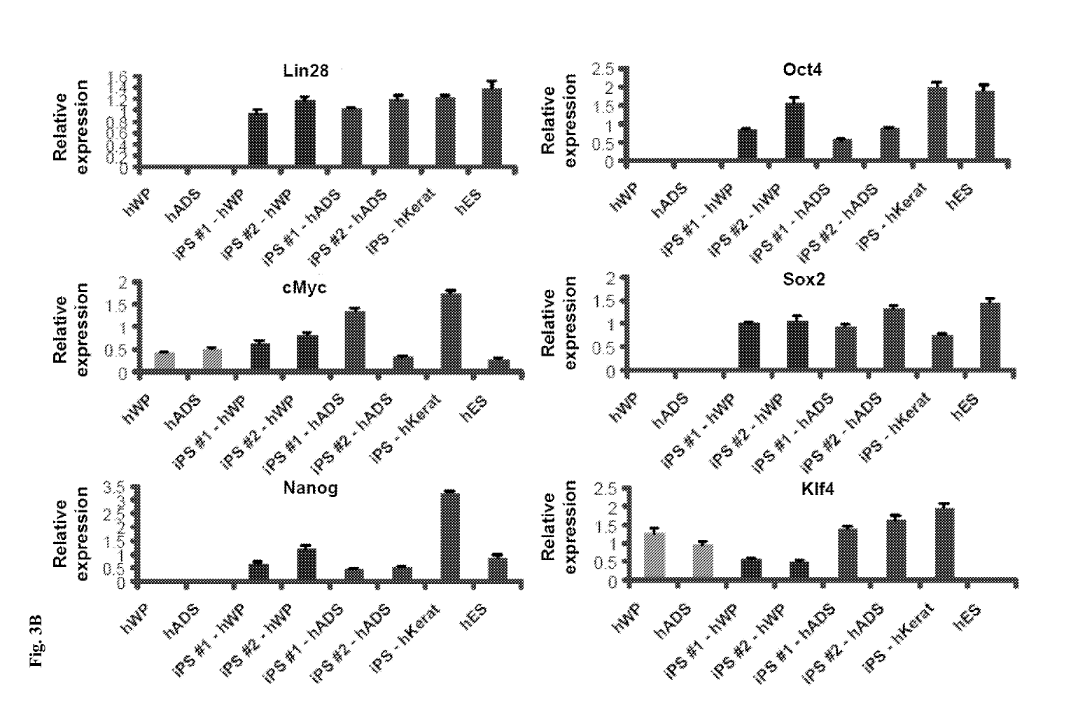

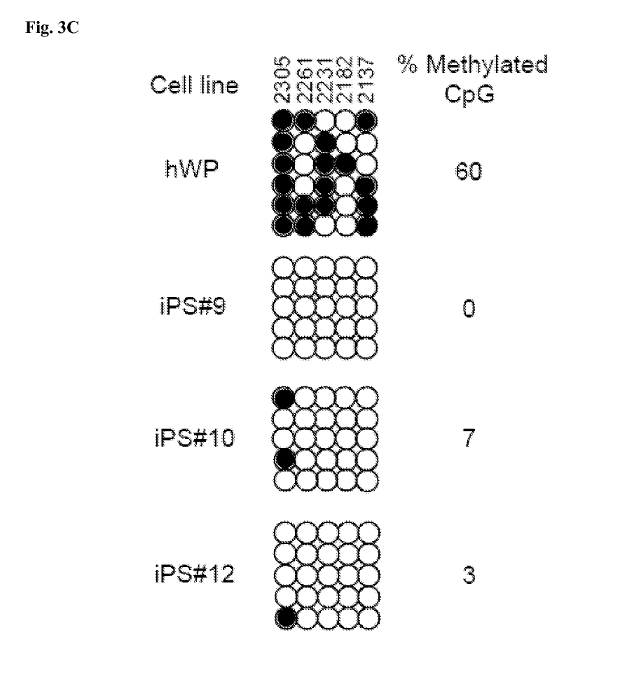

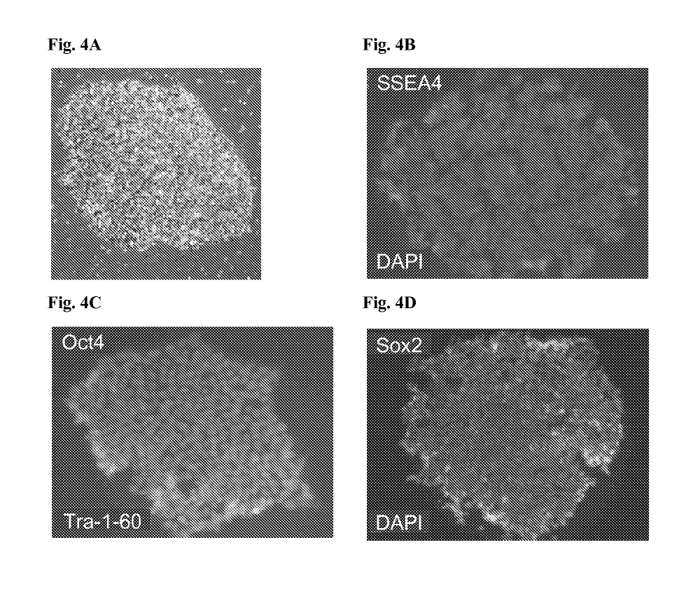

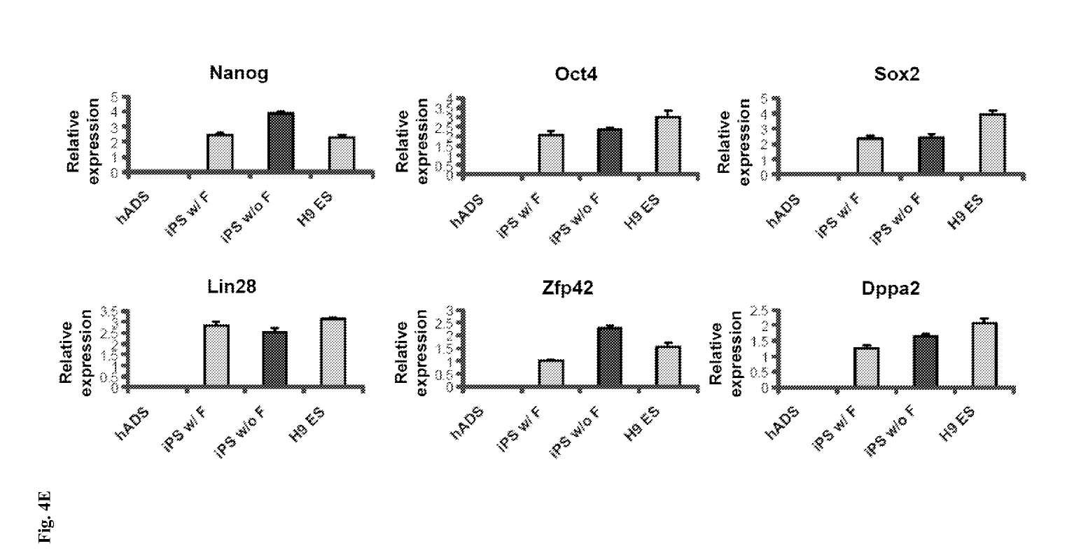

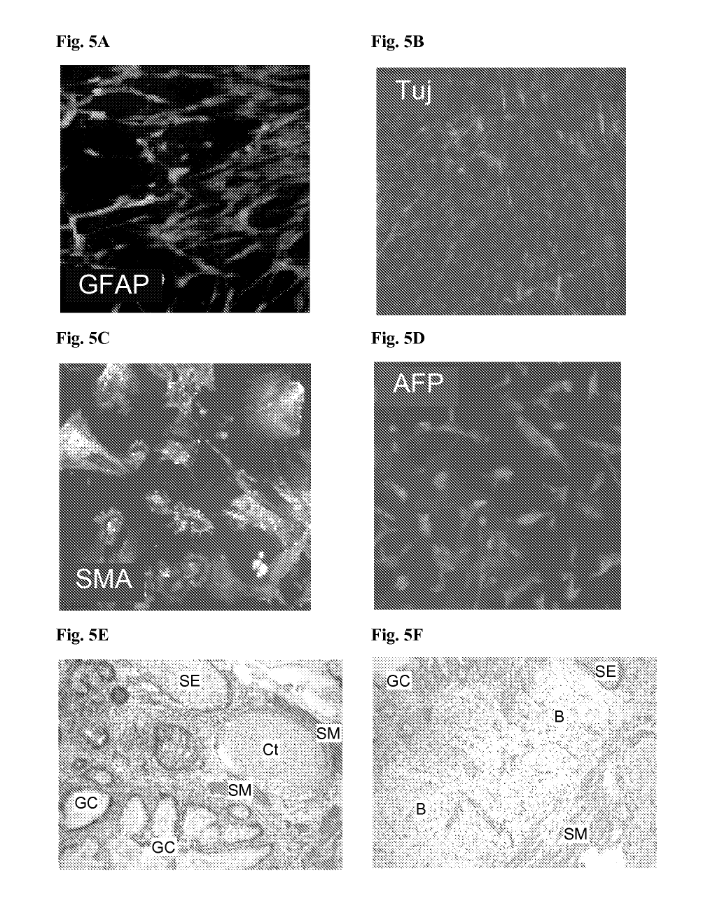

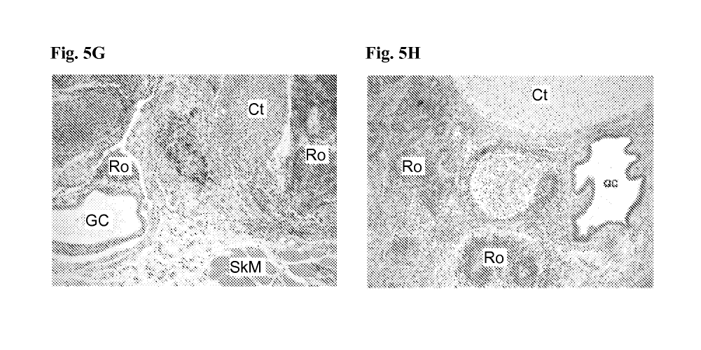

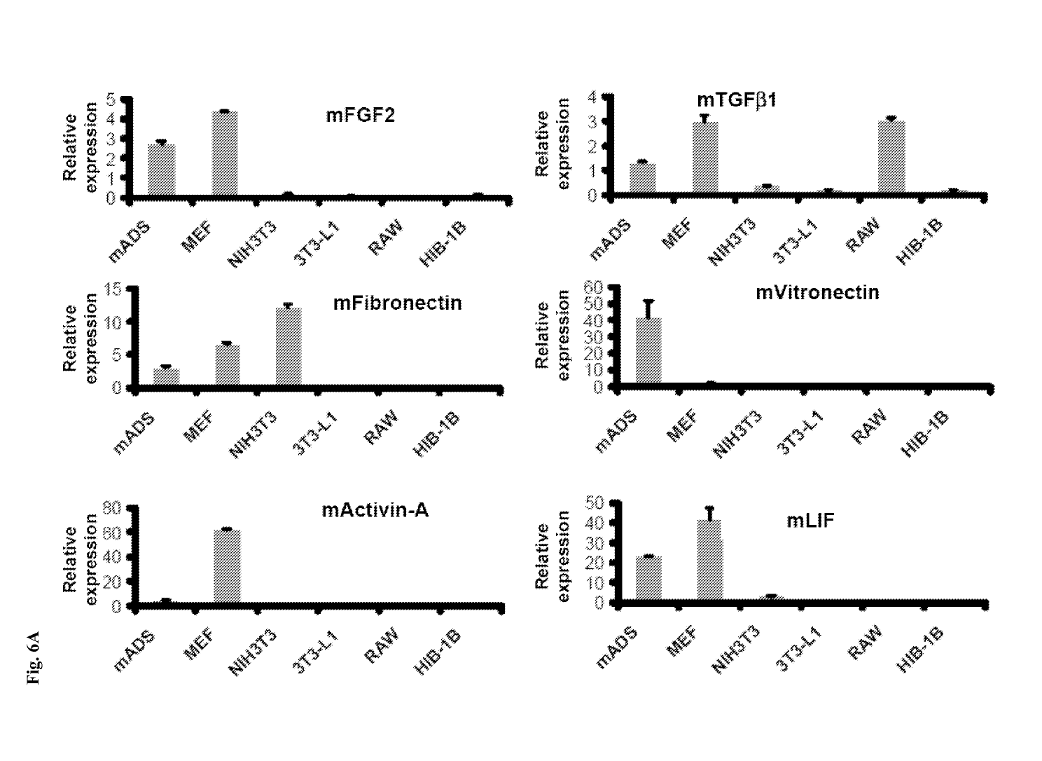

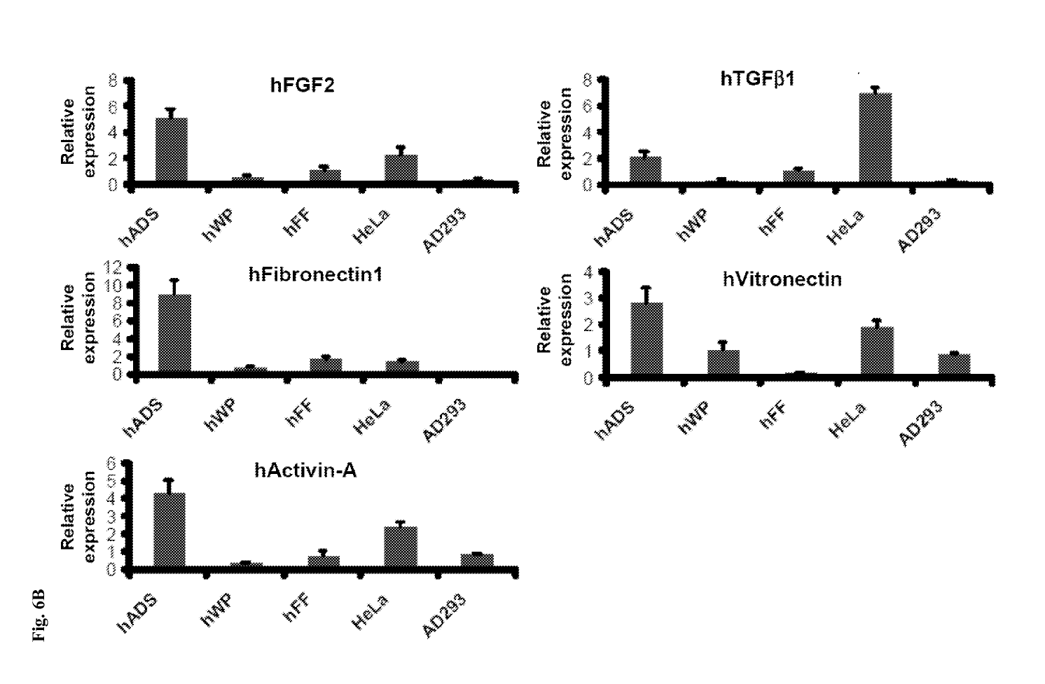

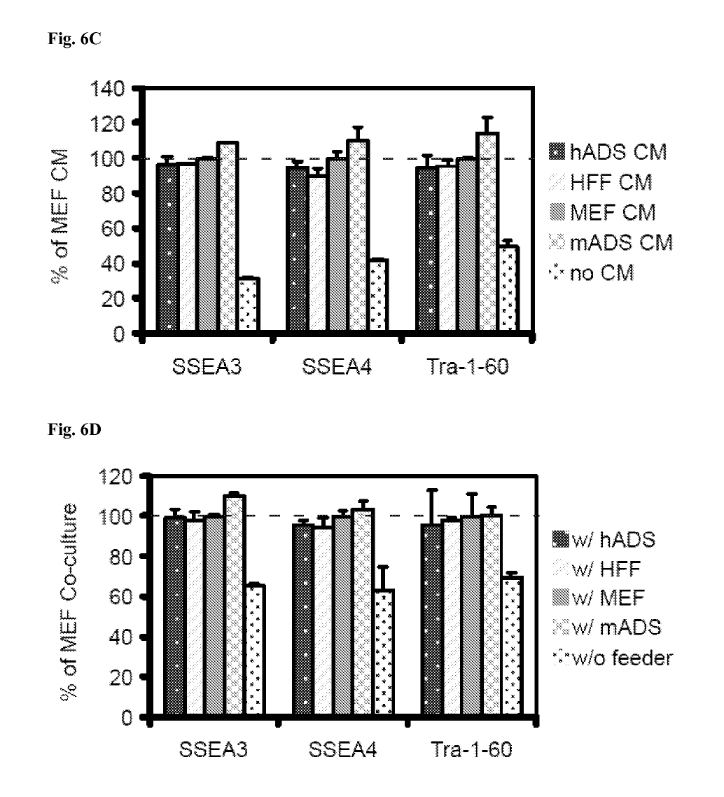

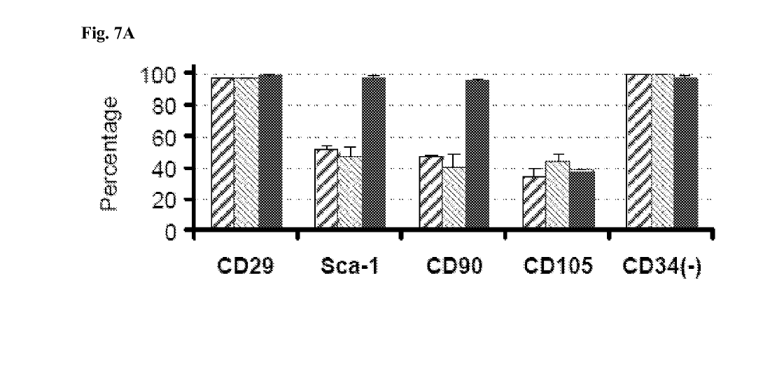

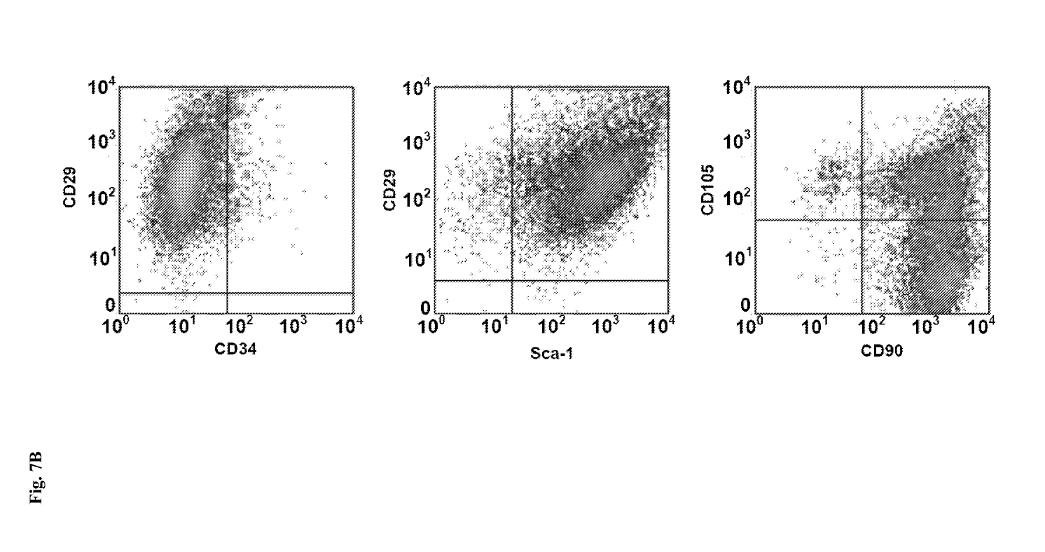

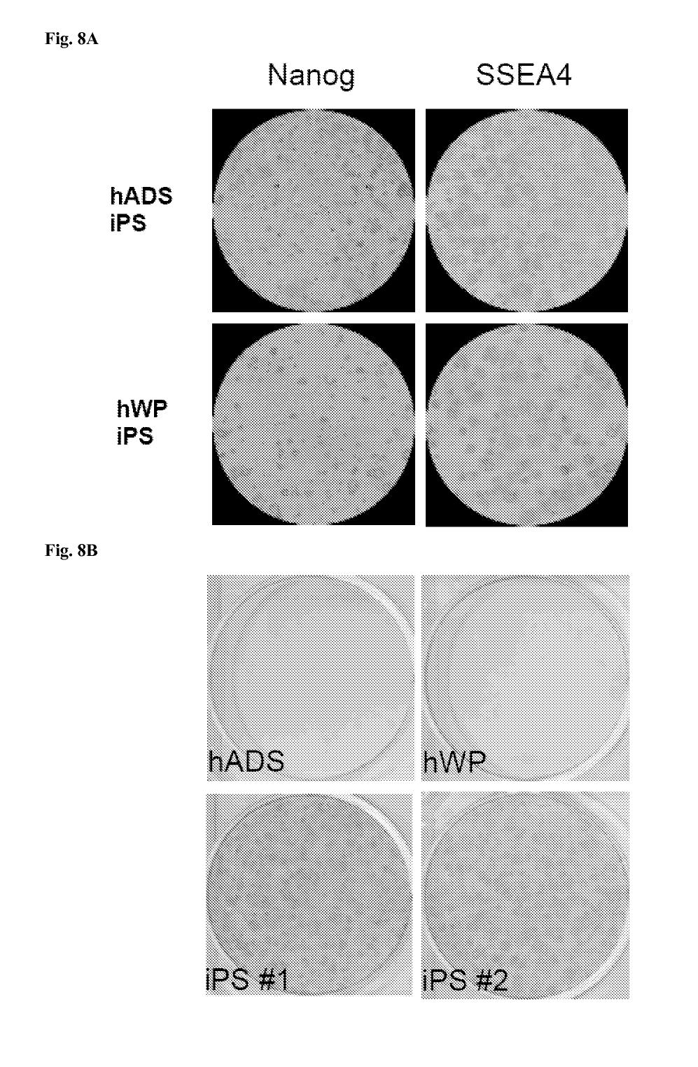

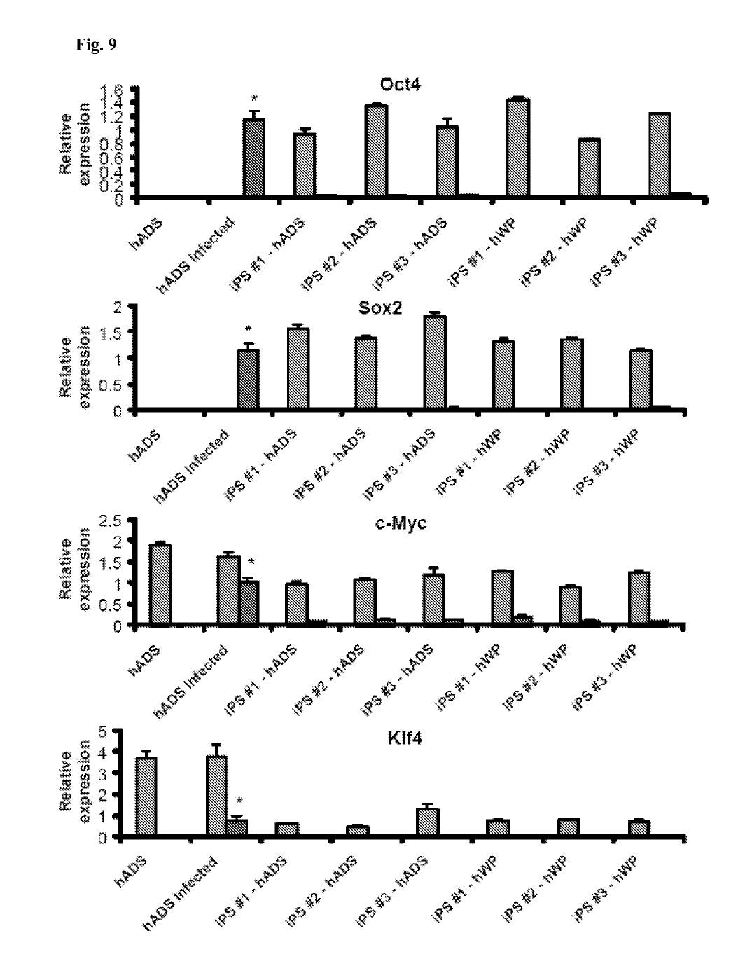

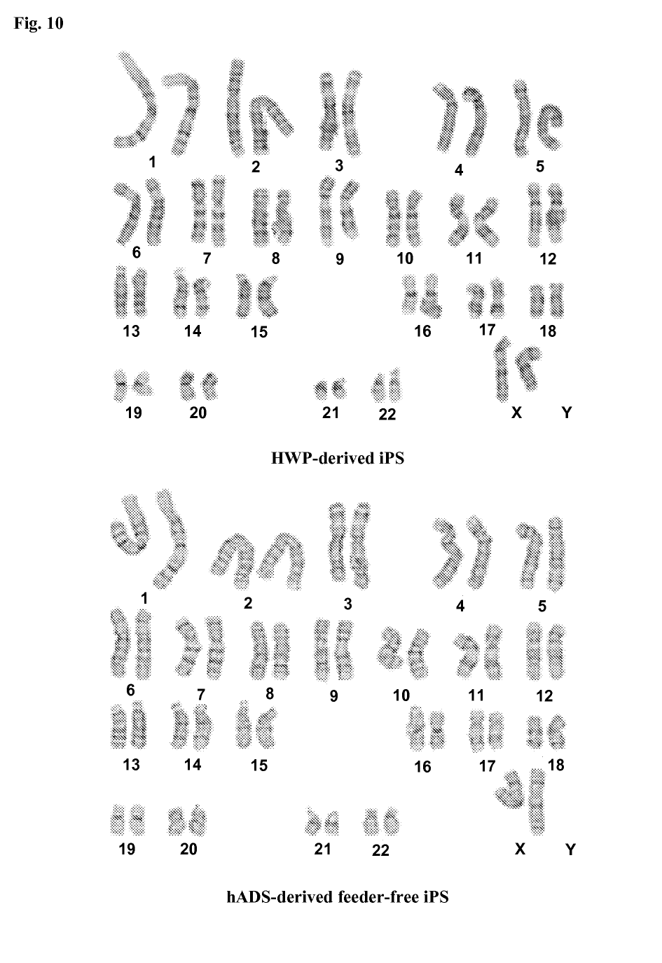

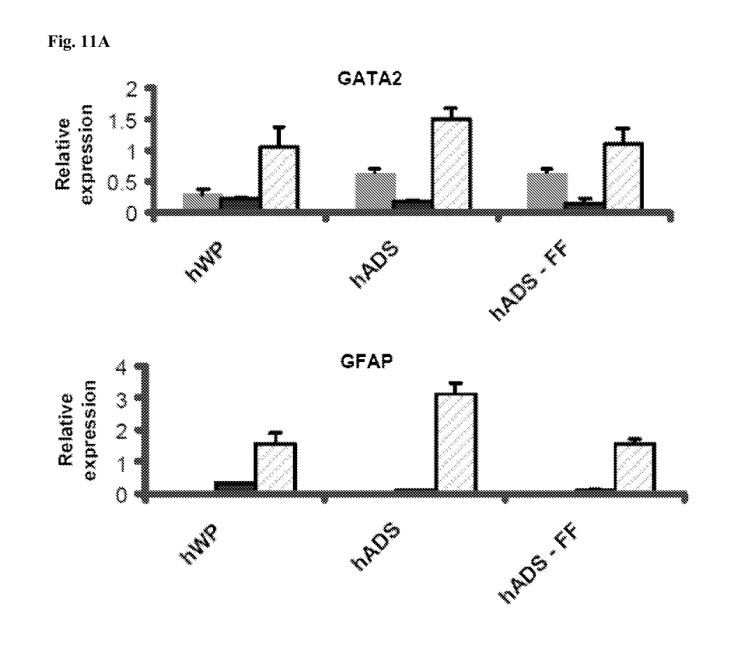

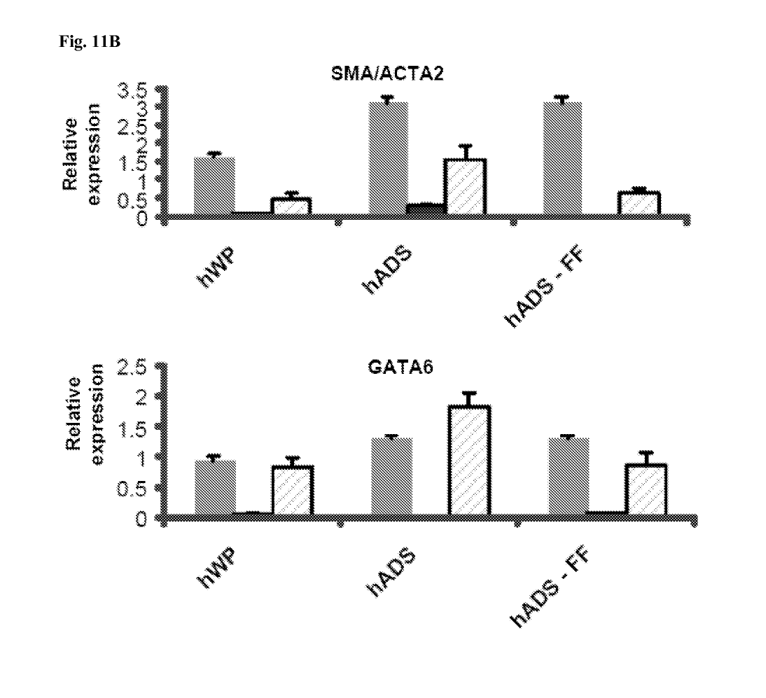

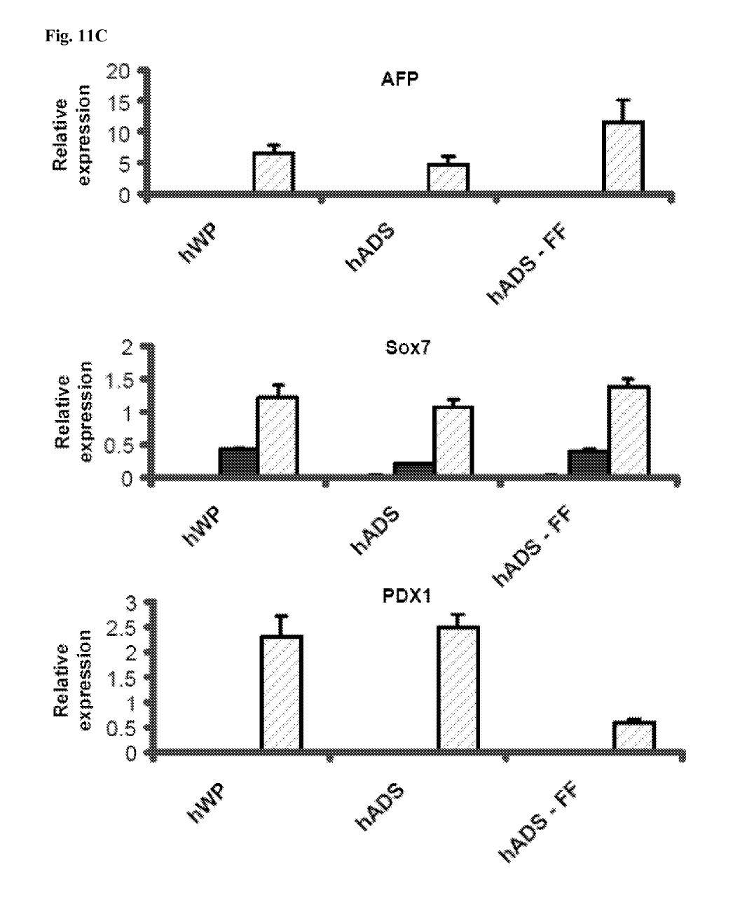

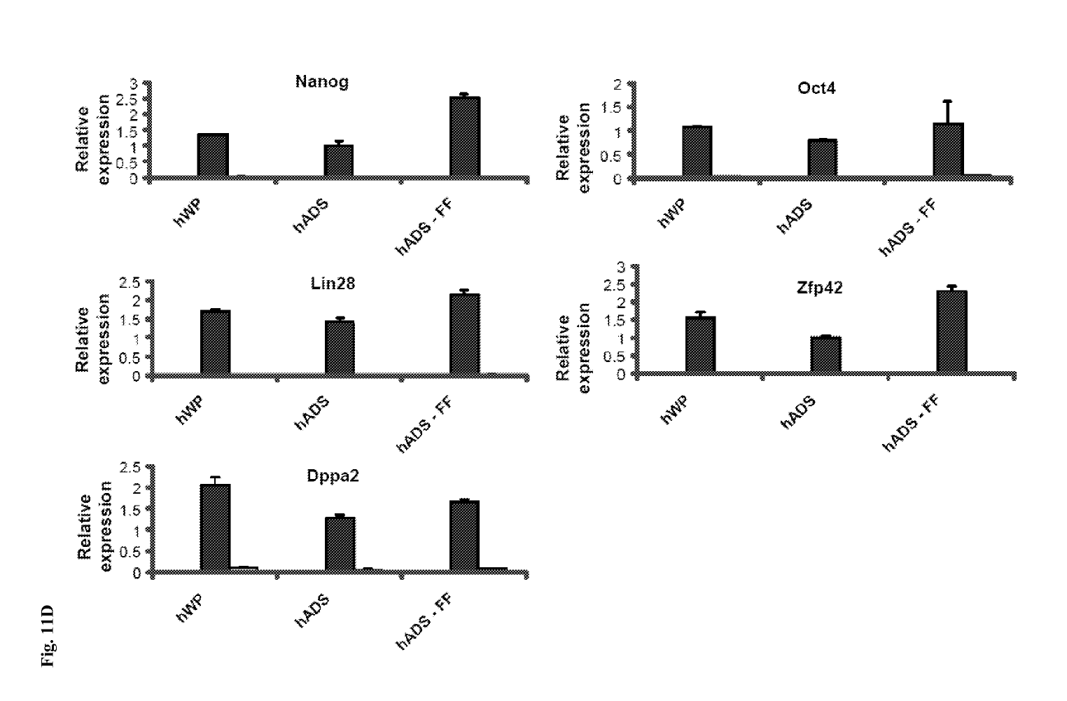

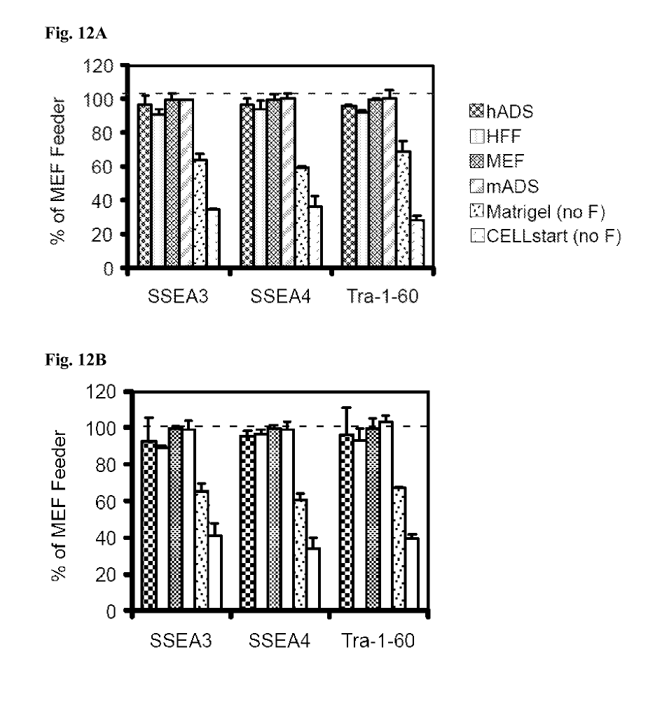

Development of hiPS lines from hADS cells (upper panels) and hWP cells (lower panels) after reprogramming factor introduction starting on day 0. (FIG. 3B) Histograms depict gene expression analysis by qPCR which shows that two independent hWP- and hADS-derived hiPS lines exhibit comparable levels of pluripotent markers to human keratinocyte-derived (hKerat) hiPS and H9 ES (hES) cells. Order (left to right): hWP, hADS, iPS#1-hWP; iPS#2-ihWP, iPS#1-hADS, iPS#2-hADS, iPS-hKerat, hES. (FIG. 3C) DNA methylation analysis of various Oct4 promoter regions indicates that somatic cells (hWP) are highly methylated whereas three independent hiPS (#9, #10, and #12) clones are hypomethylated.

FIG. 4: Characterization of human adipose-derived iPS cell lines produced in the feeder free condition. (FIG. 4A) Morphology of a hADS-derived hiPS colony developed under a completely feeder-free condition. (FIGS. 4B-4D) Representative immunofluorescence images of the feeder-free hADS-derived hiPS cells. Expression of ES cell surface antigens, SSEA4 and Tra-1-60 (FIGS. 4B and 4C), and nuclear transcription factors, Oct4 and Sox2 (FIGS. 4C and 4D), is observed. (FIG. 4E) Histograms depict qPCR analysis which indicates that feeder-free hiPS from hADS cells (iPS w/o F) express pluripotent marker genes with levels comparable to hADS-hiPS made with feeder cells (iPS w/F) or human ES cells (H9 ES). Order (left to right): hADS, iPS w/F, iPS w/o F, H9 ES. Abbreviations: (“w/”) with; (“w/o”) without.

FIG. 5: Differentiation capacities of human adipose-derived iPS cells produced in the feeder free condition. (FIGS. 5A-5D) In vitro differentiation of feeder-free hiPS-derived embryoid bodies indicates markers of ectoderm, GFAP (glial fibrillary acidic protein) and Tuj (beta III tubulin) (FIGS. 5A and 5B), of mesoderm, SMA (smooth muscle actin) (FIG. 5C), and of endoderm, AFP (alpha fetoprotein) (FIG. 5D). (FIGS. 5E-5H) Feeder-free (FIGS. 5E and 5F), hADS-derived (FIG. 5G) and hWP-derived (FIG. 5H) iPS cells exhibit in vivo differentiation capability and contribute to all three germ layers in teratomas. Ectoderm (Ro: rosette, B: brain, SE: squamous epithelium), mesoderm (Ct: cartilage, SM: smooth muscle, SkM: skeletal muscle), and endoderm (GC: goblet cells, P: pancreas).

FIG. 6: High intrinsic pluripotency-supporting capabilities of adipose-derived stem cells. (FIG. 6A) Histograms depict mouse adipose-derived stem (mADS) cells which express high levels of self-renewal supporting factors that are comparable to those of MEF by qPCR. Order (left to right): mADS, MEF, NIH3T3, 3T3-L1, RAW, HIB-1B. (FIG. 6B) Histograms depict human adipose-derived stem (hADS) cells which express higher levels of self-renewal factors than most of other human cell lines. Order (left to right): hADS, hWP, hFF, HeLa, AD293. (FIGS. 6C-6D) Histograms depict hADS-derived iPS cells which were grown for 3 passages on matrigel either in condition media (CM) taken from irradiated hADS, human foreskin fibroblasts (HFF), MEF, or mADS (FIG. 6C), or by co-culturing with these cells in Tranwell plates (FIG. 6D). Flow cytometry using antibodies against SSEA3, SSEA4, and Tra-1-60 was performed to investigate relative expression of cell surface markers. Legend for FIG. 6C: hADS CM (solid with dots); HFF CM (gray diagonal stripes); MEF CM (solid gray); mADS CM (checks); no CM (dots). Legend for FIG. 6D: w/hADS (solid with dots); w/HFF (gray diagonal stripes); w/MEF (solid gray); w/mADS (checks); w/o feeder (dots).

FIG. 7: Cell surface marker profiles (histograms) of SVF Lin⁻ and mADS cells. (FIG. 7A) Histograms depict flow cytometry analysis which indicates percentage of mADS (n=2) and SVF Lin⁻ (n=6) cells that are positive for cell surface markers (negative for CD34). Legend (abscissa): CD29, Sca-1, CD90, CD105, CD34(−). Legend (cell surface markers): mADS (thick diagonal stripes); SVF Lin— (thin diagonal stripes); SVF Lin— Sorted (solid). (FIG. 7B) SVF Lin⁻ cells were further sorted by Sca-1, and tested for cell surface markers after two passages. The percentage from n=2 samples is shown in FIG. 7A. The cells contain two populations, CD105⁺ and CD105⁻ cells, which were separated and immediately used for iPS generation.

FIG. 8: Pluripotency marker expression in human adipose-derived iPS cells. (FIG. 8A) hADS- (upper panels) and hWP-derived (lower panels) iPS cells on dishes were immunostained for Nanog (left) and SSEA4 (right) pluripotent markers. (FIG. 8B) hADS, hWP (upper panels) and two independently derived iPS lines (lower panels) were stained for alkaline phosphatase.

FIG. 9: FIG. 9 depicts histograms of silencing of exogenous gene expression and induction of endogenous genes. qPCR analysis shows that exogenous transgenes of Oct4, Sox2, c-Myc and Klf4 were effectively silenced, whereas endogenous genes were induced instead in all the derived iPS clones from hADS and hWP lines (three individual clones each line). ‘hADS infected’ is the cell 3 days after retroviral transduction. “*” indicates transgenic; other histogram entries are endogenous. Order (left to right): hADS, hADS infected, iPS#1-hADS, iPS#2-hADS, iPS#3-hADS, iPS#1-hWP, iPS#2-hWP, iPS#3-hWP.

FIG. 10: FIG. 10 depicts normal karyotype of adipose-derived hiPS lines. Chromosome stabilities of hWP-derived (left) and hADS-derived feeder-free (right) hiPS lines were investigated by karyotyping analyses. No clonal abnormalities were detected (46,XX) in hWP hiPS, in which 6 cells were karyotyped and 12 cells were analyzed. No clonal abnormalities were detected (46,XX) for hADS feeder-free hiPS as well, in which 4 cells were karyotyped and 9 cells were analyzed. Top panel: HWP-derived iPS. Bottom panel: hADS-derived feeder-free iPS.

FIG. 11: FIGS. 11A-11D depict differentiation markers in embryoid bodies formed from human adipose-derived iPS cells in ectoderm (FIG. 11A), mesoderm (FIG. 11B), endoderm (FIG. 11C). FIG. 11D depicts pluripotency markers. qPCR analysis was performed in somatic cells, iPS cells and embryoid bodies from either hWP or hADS to examine differentiation markers of the three germ layers: GATA2 and GFAP for ectoderm (FIG. 11A); SMA/ACTA2 and GATA6 for mesoderm (FIG. 11B); AFP, Sox? and PDX1 for endoderm (FIG. 11C) and pluripotency markers Nanog, Oct4, Lin28, Zfp42 and Dppa2 (FIG. 11D). Legend (FIGS. 11A-11D): somatic (gray); iPS (solid black); EB (diagonal stripes).

FIG. 12: Adipose-derived stem cells serve as feeder layers for heterologous hiPS lines. hWP-derived (FIG. 12A) and hKeratinocyte-derived (FIG. 12B) hiPS cells were cultured for 7-10 passages on irradiated hADS, HFF, MEF, or mADS as feeder layers. Control cells from MEF layers were cultured for the last two passages on matrigel or CELLstart without feeders (no F). Cell surface markers were then examined by flow cytometry. Legend (left to right in FIGS. 12A-12B): hADS (loose checkered); HFF (vertical gray stripes); MEF (tight checkered); mADS (diagonal stripes); Matrigel (no F) (open with irregular spaced dots); CELLstart (no F) (open with regular spaced dots).

### DETAILED DESCRIPTION OF THE INVENTION

### I. Definitions

The following definitions are provided to facilitate understanding of certain terms used frequently herein and are not meant to limit the scope of the present disclosure.

“Nucleic acid” refers to deoxyribonucleotides or ribonucleotides and polymers thereof in either single- or double-stranded form, and complements thereof. A person having ordinary skill in the art will immediately understand that a nucleic acid encoding one or more proteins (e.g. cMYC, KLF4, OCT4 and SOX2) that is transfected into a cell will also include any necessary sequences (e.g. promoter sequences, etc.) to allow the proteins encoded by the nucleic acid to be expressed in the cell.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “identical” or percent “identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www(dot)ncbi(dot)nlm(dot)nih(dot)gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The phrase “stringent hybridization conditions” refers to conditions under which a probe will hybridize to its target sequence, typically in a complex mixture of nucleic acids, but to not other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology--Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5x SSC, and 1% SDS, incubating at 42° C., or, 5x SSC, 1% SDS, incubating at 65° C., with wash in 0.2x SSC, and 0.1% SDS at 65° C.

A variety of methods of specific DNA and RNA measurement that use nucleic acid hybridization techniques are known to those of skill in the art (see, Sambrook, supra). Some methods involve electrophoretic separation (e.g., Southern blot for detecting DNA, and Northern blot for detecting RNA), but measurement of DNA and RNA can also be carried out in the absence of electrophoretic separation (e.g., by dot blot).

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman and molecular beacon probes can be used to monitor amplification reaction products, e.g., in real time.

The word “polynucleotide” refers to a linear sequence of nucleotides. The nucleotides can be ribonucleotides, deoxyribonucleotides, or a mixture of both. Examples of polynucleotides contemplated herein include single and double stranded DNA, single and double stranded RNA (including miRNA), and hybrid molecules having mixtures of single and double stranded DNA and RNA.

The words “protein”, “peptide”, and “polypeptide” are used interchangeably to denote an amino acid polymer or a set of two or more interacting or bound amino acid polymers.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “episomal” refers to the extra-chromosomal state of a plasmid in a cell. Episomal plasmids are nucleic acid molecules that are not part of the chromosomal DNA and replicate independently thereof.

A “cell culture” is a population of cells residing outside of an organism. These cells are optionally primary cells isolated from a cell bank, animal, or blood bank, or secondary cells that are derived from one of these sources and have been immortalized for long-lived in vitro cultures.

A “stem cell” is a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair .

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells.

“Pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

The term “reprogramming” refers to the process of dedifferentiating a non-pluripotent cell into a cell exhibiting pluripotent stem cell characteristics.

The term “treating” means ameliorating, suppressing, eradicating, and/or delaying the onset of the disease being treated.

An “induced pluripotent stem cell” refers to a pluripotent stem cell artificially derived from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells. An adult stem cell is an undifferentiated cell found throughout the body after embryonic development. Adult stem cells multiply by cell division to replenish dying cells and regenerate damaged tissue. Adult stem cells have the ability to divide and create another cell like itself and also divide and create a cell more differentiated than itself. Even though adult stem cells are associated with the expression of pluripotency markers such as Rex1, Nanog, Oct4 or Sox2, they do not have the ability of pluripotent stem cells to differentiate into the cell types of all three germ layers. Adult stem cells have a limited potency to self renew and generate progeny of distinct cell types. Without limitation, an adult stem cell can be a hematopoietic stem cell, a cord blood stem cell, a mesenchymal stem cell, an epithelial stem cell, a skin stem cell or a neural stem cell. A tissue specific progenitor refers to a cell devoid of self-renewal potential that is committed to differentiate into a specific organ or tissue. A primary cell includes any cell of an adult or fetal organism apart from egg cells, sperm cells and stem cells. Examples of useful primary cells include, but are not limited to, skin cells, bone cells, blood cells, cells of internal organs and cells of connective tissue. A secondary cell is derived from a primary cell and has been immortalized for long-lived in vitro cell culture.

An “adipose-derived stem cell” as used herein is a stem cell derived from adipose tissue. The term includes stem cells derived from progenitor cells, mesenchymal stem cells, pre-adipocyte cells (e.g. white pre-adipocytes) and hematopoietic cells residing in adipose tissue.

A “somatic cell” is a cell forming the body of an organism. Somatic cells include cells making up organs, skin, blood, bones and connective tissue in an organism, but not germline cells.

The term “transfection” or “transfecting” is defined as a process of introducing nucleic acid molecules to a cell by non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell.

Such a selection advantage may be a resistance towards a certain toxin that is presented to the cell. Expression of a transfected gene can further be accomplished by transposon-mediated insertion into to the host genome. During transposon-mediated insertion the gene is positioned between two transposon linker sequences that allow insertion into the host genome as well as subsequent excision.

An “OCT4 protein” as referred to herein includes any of the naturally-occurring forms of the Octomer 4 transcription factor, or variants thereof that maintain Oct4 transcription factor activity (e.g. within at least 50%, 80%, 90%, 95%, 96%, 97%, 98%, 99% or 100% activity compared to Oct4). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Oct4 polypeptide (e.g. SEQ ID NO:67, SEQ ID NO:68 or SEQ ID NO:69). In other embodiments, the Oct4 protein is the protein as identified by the NCBI reference gi:42560248 corresponding to isoform 1 (SEQ ID NO:67), gi:116235491 and gi:291167755 corresponding to isoform 2 (SEQ ID NO:68 and SEQ ID NO:69).

A “Sox2 protein” as referred to herein includes any of the naturally-occurring forms of the Sox2 transcription factor, or variants thereof that maintain Sox2 transcription factor activity (e.g. within at least 50%, 80%, 90%, 95%, 96%, 97%, 98%, 99% or 100% activity compared to Sox2). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Sox2 polypeptide (e.g. SEQ ID NO:70). In other embodiments, the Sox2 protein is the protein as identified by the NCBI reference gi:28195386 (SEQ ID NO:70).

A “KLF4 protein” as referred to herein includes any of the naturally-occurring forms of the KLF4 transcription factor, or variants thereof that maintain KLF4 transcription factor activity (e.g. within at least 50%, 80%, 90%, 95%, 96%, 97%, 98%, 99% or 100% activity compared to KLF4). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring KLF4 polypeptide (e.g. SEQ ID NO:71). In other embodiments, the KLF4 protein is the protein as identified by the NCBI reference gi:194248077 (SEQ ID NO:71).

A “cMYC protein” as referred to herein includes any of the naturally-occurring forms of the cMyc transcription factor, or variants thereof that maintain cMyc transcription factor activity (e.g. within at least 50%, 80%, 90%, 95%, 96%, 97%, 98%, 99% or 100% activity compared to cMyc). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring cMyc polypeptide (e.g. SEQ ID NO:72). In other embodiments, the cMyc protein is the protein as identified by the NCBI reference gi:71774083 (SEQ ID NO:72).

The term “xeno-free,” as used herein, refers to the absence of non-human animal derived substances. A non-human animal derived substance refers to a substance derived from an animal that is not human, such as mouse, rat, rabbit, monkey or other animal. Non-human animal derived substances include biomolecules (e.g. proteins, nucleic acid, etc.), cells, and the like. For example, a “xeno-free liquid” is a liquid that does not contain non-human animal derived substances. In some embodiments, xeno-free refers to the absence of xenobiotics. Thus, where a xeno-freee environment is employed, a person having ordinary skill in the art will understand that the employed adipose-derived stem cell is a human adipose-derived stem cell and the employed or formed adipose-derived induced pluripotent stem cell is a human adipose-derived induced pluripotent stem cell.

The term “feeder-free,” refers to the absence of feeder cells. The term “feeder cell” is generally well know in the art and includes all cells used to support the propagation of stem cells during the process of reprogramming. Feeder cells may be irradiated prior to being co-cultured with the cells being reprogrammed in order to avoid the feeder cells to overgrow the cells undergoing reprogramming. Feeder cells produce growth factors that support cells during the process of reprogramming and also provide a layer physical support for the reprogrammed cells to attach to. Examples of feeder cells include fibroblasts, splenocytes, macrophages and thymocytes.

Where appropriate the expanding transfected adipose-derived stem cell may be subjected to a process of selection. A process of selection may include a selection marker introduced into a adipose-derived stem cell upon transfection. A selection marker may be a gene encoding for a polypeptide with enzymatic activity. The enzymatic activity includes, but is not limited to, the activity of an acetyltransferase and a phosphotransferase. In some embodiments, the enzymatic activity of the selection marker is the activity of a phosphotransferase. The enzymatic activity of a selection marker may confer to a transfected adipose-derived stem cell the ability to expand in the presence of a toxin. Such a toxin typically inhibits cell expansion and/or causes cell death. Examples of such toxins include, but are not limited to, hygromycin, neomycin, puromycin and gentamycin. In some embodiments, the toxin is hygromycin. Through the enzymatic activity of a selection maker a toxin may be converted to a non-toxin, which no longer inhibits expansion and causes cell death of a transfected adipose-derived stem cell. Upon exposure to a toxin a cell lacking a selection marker may be eliminated and thereby precluded from expansion.

Identification of the induced pluripotent stem cell may include, but is not limited to the evaluation of the afore mentioned pluripotent stem cell characteristics. Such pluripotent stem cell characteristics include without further limitation, the expression or non-expression of certain combinations of molecular markers. Further, cell morphologies associated with pluripotent stem cells are also pluripotent stem cell characteristics.

### II. Compositions and Methods for Preparing Adipose-Derived Induced Pluripotent Stem Cells

In another aspect, a method for preparing an adipose-derived induced pluripotent stem cell is provided. The method includes transfecting an adipose-derived stem cell with a nucleic acid encoding an OCT4 protein and a nucleic acid encoding a SOX2 protein to form a transfected adipose-derived stem cell. The transfected adipose-derived stem cell is allowed to divide (e.g. grow and/or differentiate) thereby forming the adipose-derived induced pluripotent stem cell.

The method may further include transfecting the adipose-derived stem cell with a nucleic acid encoding a KLF4 protein and a nucleic acid encoding a SOX2 protein. Thus, in another aspect, a method for preparing an adipose-derived induced pluripotent stem cell is provided. The method includes transfecting an adipose-derived stem cell with a nucleic acid encoding a cMYC protein, a nucleic acid encoding an OCT4 protein, a nucleic acid encoding a KLF4 protein and a nucleic acid encoding a SOX2 protein to form a transfected adipose-derived stem cell. The transfected adipose-derived stem cell is allowed to divide (e.g. grow and/or differentiate) thereby forming the adipose-derived induced pluripotent stem cell. In some embodiments, the adipose-derived stem cell is a pre-adipocyte stem cell. In some embodiments the adipose-derived stem cell is a human adipose-derived stem cell and the adipose-derived induced pluripotent stem cell is a human adipose-derived induced pluripotent stem cell.

In some embodiments, where a cell is transfected with a nucleic acid encoding a protein as described herein, the transfection is performed with only the recited nucleic acids encoding the recited proteins (e.g. KLF4, OCT4, SOX2 and/or cMYC) in the absence of other proteins known in the art to be useful in forming an induced pluripotent stem cell (e.g. in the absence of a nucleic acid encoding a LTN28 protein or a NANOG protein).

The transfected adipose-derived stem cell is typically allowed to divide in an environment with appropriate cellular nutrients. The environment may be a liquid environment, a solid environment and/or a semisolid environment (e.g. agar, gel etc.). A growth medium may be employed. A “growth medium” as used herein, is used according to its generally accepted meaning in the art. A growth medium (also referred to in the art and herein as a “culture medium”) includes liquids or gels designed to support the growth (e.g. division, differentiation, etc.) of cells.

In some embodiments, the transfected adipose-derived stem cell is allowed to divide in the absence of feeder cells (i.e. a feeder-free environment). For example, a feeder-free growth medium may be employed, including a feeder-free liquid growth medium or a feeder-free gel medium. The environment may also be a xeno-free environment. For example, a xeno-free growth medium may be employed, including a xeno-free liquid growth medium or a xeno-free gel medium. Thus, the environment may also be a feeder-free and xeno-free environment.

In another aspect, a method of culturing (e.g. maintaining or growing) an induced pluripotent stem cell is provided. The method includes contacting the induced pluripotent stem cell with a growth medium. The growth medium includes an adipose-derived induced pluripotent stem cell. The method further comprises allowing the induced pluripotent stem cell to divide in the presence of the growth medium. In some embodiments, the growth medium is also a xeno-free growth medium, such as a xeno-free liquid growth medium and/or xeno-free gel growth medium. Thus, in some embodiments, the culturing occurs in a xeno-free environment. While the growth medium may include one or more adipose-derived induced pluripotent stem cells, the growth medium may not include any other types of cells (e.g. other feeder cells). The adipose-derived induced pluripotent stem cell may be produced using the methods described herein. For example, the method may include transfecting an adipose-derived stem cell with a nucleic acid encoding a cMYC protein, a nucleic acid encoding an OCT4 protein, a nucleic acid encoding a KLF4 protein and a nucleic acid encoding a SOX2 protein to form a transfected adipose-derived stem cell. The transfected adipose-derived stem cell is allowed to divide thereby forming said adipose-derived induced pluripotent stem cell.

In another aspect, a xeno-free liquid composition is provided. The xeno-free liquid composition is prepared by a process comprising transfecting an adipose-derived stem cell with a nucleic acid encoding an OCT4 protein and a nucleic acid encoding a SOX2 protein to form a transfected adipose-derived stem cell. In some embodiments, the xeno-free liquid composition is prepared by a process comprising transfecting an adipose-derived stem cell with a nucleic acid encoding a cMYC protein, a nucleic acid encoding an OCT4 protein, a nucleic acid encoding a KLF4 protein and a nucleic acid encoding a SOX2 protein to form a transfected adipose-derived stem cell. The transfected adipose-derived stem cell is allowed to divide in a xeno-free liquid. The xeno-free liquid is isolated (e.g. separated from the adipose derived stem cell) thereby preparing the xeno-free liquid composition. In some embodiments, the xeno-free liquid composition is a xeno-free liquid growth medium, or forms part of a xeno-free liquid growth medium or xeno-free gel growth medium. The xeno-free liquid composition, xeno-free gel growth medium and xeno-free liquid growth medium may also be feeder-free (i.e. a feeder-free and xeno-free liquid composition, feeder-free and xeno-free gel growth medium and feeder-free and xeno-free liquid growth medium, respectively).

In another aspect, a method of culturing (e.g. maintaining or growing) an induced pluripotent stem cell is provided. The method includes contacting the induced pluripotent stem cell with a xeno-free growth medium. The xeno-free growth medium includes the xeno-free liquid composition prepared by the methods provided herein. The induced pluripotent stem cell is allowed to divide in the presence of the growth medium. The xeno-free growth medium may be a xeno-free liquid growth medium or a xeno-free gel growth medium. The xeno-free gel growth medium may also be feeder-free. Thus, in some embodiments, the xeno-free growth medium is a feeder-free and xeno-free growth medium, such as a feeder-free and xeno-free gel growth medium or a feeder-free and xeno-free liquid growth medium. The induced pluripotent stem cell may be an adipose-derived induced pluripotent stem cell. The adipose-derived induced pluripotent stem cell may be produced using the methods described herein. For example, the method may include transfecting an adipose-derived stem cell with a nucleic acid encoding a cMYC protein, a nucleic acid encoding an OCT4 protein, a nucleic acid encoding a KLF4 protein and a nucleic acid encoding a SOX2 protein to form a transfected adipose-derived stem cell. The transfected adipose-derived stem cell is allowed to divide thereby forming said adipose-derived induced pluripotent stem cell.

Where the methods or compositions provided herein employ an adipose-derived stem cell, the adipose-derived stem cell may be a pre-adipocyte stem cell or an adipose-derived mesenchymal stem cells. In some embodiments, the adipose-derived stem cells is not replicating. In one embodiment, the adipose-derived stem cell is a white pre-adipocyte.

Where the methods provided herein allow a transfected adipose-derived stem cell to divide, the dividing may occurs in the absence of feeder cells (i.e. a feeder-free environment) and/or a xeno-free environment as described above..

Allowing the transfected adipose-derived stem cell to divide and thereby forming the adipose-derived induced pluripotent stem cell may include expansion of the adipose-derived stem cell after transfection, optional selection for transfected cells and identification of pluripotent stem cells. Expansion as used herein includes the production of progeny cells by a transfected adipose-derived stem cell in containers and under conditions well know in the art. Expansion may occur in the presence of suitable media and cellular growth factors. Cellular growth factors are agents, which cause cells to migrate, differentiate, transform or mature and divide. They are polypeptides, which can usually be isolated from various normal and malignant mammalian cell types. Some growth factors can also be produced by genetically engineered microorganisms, such as bacteria (E. coli) and yeasts. Cellular growth factors may be supplemented to the medium. Examples of cellular growth factors include, but are not limited to, FGF, bFGF2, and EGF.

Where appropriate the expanding adipose-derived stem cell may be subjected to a process of selection. A process of selection may include a selection marker introduced into a adipose-derived stem cell upon transfection. A selection marker may be a gene encoding for a polypeptide with enzymatic activity. The enzymatic activity includes, but is not limited to, the activity of an acetyltransferase and a phosphotransferase. In some embodiments, the enzymatic activity of the selection marker is the activity of a phosphotransferase. The enzymatic activity of a selection marker may confer to a transfected adipose-derived stem cell the ability to expand in the presence of a toxin. Such a toxin typically inhibits cell expansion and/or causes cell death. Examples of such toxins include, but are not limited to, hygromycin, neomycin, puromycin and gentamycin. In some embodiments, the toxin is hygromycin. Through the enzymatic activity of a selection maker a toxin may be converted to a non-toxin, which no longer inhibits expansion and causes cell death of a transfected adipose-derived stem cell. Upon exposure to a toxin a cell lacking a selection marker may be eliminated and thereby precluded from expansion.

Identification of the adipose-derived induced pluripotent stem cell may include, but is not limited to the evaluation of the aforementioned pluripotent stem cell characteristics. Such pluripotent stem cell characteristics include without further limitation, the expression or non-expression of certain combinations of molecular markers. Further, cell morphologies associated with pluripotent stem cells are also pluripotent stem cell characteristics.

In another aspect, an adipose-derived induced pluripotent stem is provided that is prepared according to methods provided herein.

In another aspect, an adipose-derived stem cell including a nucleic acid encoding a cMYC protein, a nucleic acid encoding an OCT4 protein, a nucleic acid encoding a KLF4 protein and a nucleic acid encoding a SOX2 protein is provided. The cMYC protein, OCT4 protein, KLF4 protein and SOX2 protein may be encoded within one, two, three or four nucleic acids. The nucleic acid(s) encoding the cMYC protein, OCT4 protein, KLF4 protein and SOX2 protein are exogenous to the adipose derived-stem cell.

In one aspect, an adipose-derived stem cell including a nucleic acid encoding an OCT4 protein and a nucleic acid encoding a SOX2 protein is provided. In some embodiments the adipose-derived stem cell consists essentially of a nucleic acid encoding an OCT4 protein and a nucleic acid encoding a SOX2 protein. Where an adipose-derived stem cell “consists essentially of” a nucleic acid encoding an OCT4 protein and a nucleic acid encoding a SOX2 protein, the adipose-derived stem cell does not include nucleic acids encoding other transcription factors known to be useful in iPS cell formation. In some embodiments, the adipose-derived stem cell does not include nucleic acids encoding other transcription factors. In other embodiments, the adipose-derived stem cell does not include nucleic acids encoding other protein expressing genes.

In another aspect, a method for producing a somatic cell is provided. The method includes contacting an adipose-derived induced pluripotent stem cell with cellular growth factors. The adipose-derived induced pluripotent stem cell is allowed to divide, thereby forming the somatic cell.

In some embodiments, the adipose-derived induced pluripotent stem cell is prepared in accordance with the methods provided herein. For example, the method may include transfecting an adipose-derived stem cell with a nucleic acid encoding a cMYC protein, a nucleic acid encoding an OCT4 protein, a nucleic acid encoding a KLF4 protein and a nucleic acid encoding a SOX2 protein to form a transfected adipose-derived stem cell. The transfected adipose-derived stem cell is allowed to divide thereby forming said adipose-derived induced pluripotent stem cell. As disclosed above, the transfected adipose-derived stem cell may allowed to divide in the absence of feeder cells (i.e. a feeder-free environment) and/or in a xeno-free environment. In some embodiments, the adipose-derived pluripotent stem cell is transfected with a nucleic acid encoding an OCT4 protein and a nucleic acid encoding a SOX2 protein to form a transfected adipose derived stem cell. In other embodiments, the adipose-derived stem cell is not transfected with an additional nucleic acid encoding a cMYC protein, a LIN28 protein, a NANOG protein or a KLF4 protein.

In another aspect, a method of treating a mammal in need of tissue repair is provided. The method includes administering an adipose-derived induced pluripotent stem to the mammal. The adipose-derived induced pluripotent stem cell is allowed to divide and differentiate into somatic cells in the mammal, thereby providing tissue repair in the mammal.

In some embodiments, the adipose-derived induced pluripotent stem cell is prepared in accordance with the methods provided herein. For example, the method may include transfecting an adipose-derived stem cell with a nucleic acid encoding a cMYC protein, a nucleic acid encoding an OCT4 protein, a nucleic acid encoding a KLF4 protein and a nucleic acid encoding a SOX2 protein to form a transfected adipose-derived stem cell. The transfected adipose-derived stem cell is allowed to divide thereby forming said adipose-derived induced pluripotent stem cell. As disclosed above, the transfected adipose-derived stem cell may allowed to divide in the absence of feeder cells (i.e. a feeder-free environment) and/or in a xeno-free environment. In other embodiments, the transfected adipose-derived stem cell is allowed to divide in the absence of feeder cells. In some embodiments, the adipose-derived pluripotent stem cell is transfected with a nucleic acid encoding an OCT4 protein and a nucleic acid encoding a SOX2 protein to form a transfected adipose derived stem cell. In other embodiments, the adipose-derived stem cell is not transfected with an additional nucleic acid encoding a cMYC protein, a LIN28 protein, a NANOG protein or a KLF4 protein.

In some embodiments of the methods and compositions provided herein, the adipose-derived stem cell is a human adipose-derived stem cell and the adipose-derived induced pluripotent stem cell is a human adipose-derived induced pluripotent stem cell.

### EXAMPLES

### Introduction

In industrialized countries, where liposuction procedures are common, adipose tissue is a virtually unlimited resource. While adipose tissue is comprised of heterogeneous cell populations, it is also an abundant source of progenitor and mesenchymal stem cells ^{(1,2)}. As many as 1% of adipose cells are estimated to be mesenchymal stem cells, compared to the 0.001% to 0.002% found in bone marrow, currently a common source of stem cells ⁽¹⁾. These adipose-residing stem cells have a large potential for self-renewal, while also maintaining the ability to become a limited repertoire of cell types such as adipocytes, myocytes, osteoblasts and chondrocytes. The remaining fat tissue consists of mature adipocytes, pre-adipocytes, endothelial cells, pericytes, and hematopoietic cells. Unlike bone marrow, biopsies of fat tissue can be obtained by a relatively safe and popular liposuction procedure, one of the top plastic surgeries performed in the United States in 2007 (American Society for Aesthetic Plastic Surgery*). We hypothesized that the self-renewal and multipotent properties of adipose-derived progenitor and stem cells would make them ideal candidates for the generation of induced pluripotent stem (iPS) cells by transduction with four standard reprogramming factors, cMyc, Klf4, Oct4 and Sox2 ⁽³⁻⁶⁾. Here we describe the successful production of iPS cells both from human and mouse adipose-derived cells. Unexpectedly, we found that these cells are also capable of reprogramming into iPS in the feeder- and xeno-free conditions. Adipose-derived stem cells exhibit intrinsic expression of self-renewal supporting factors and can effectively serve as feeder layers of their own or independent pluripotent cells.

### Results and Discussion

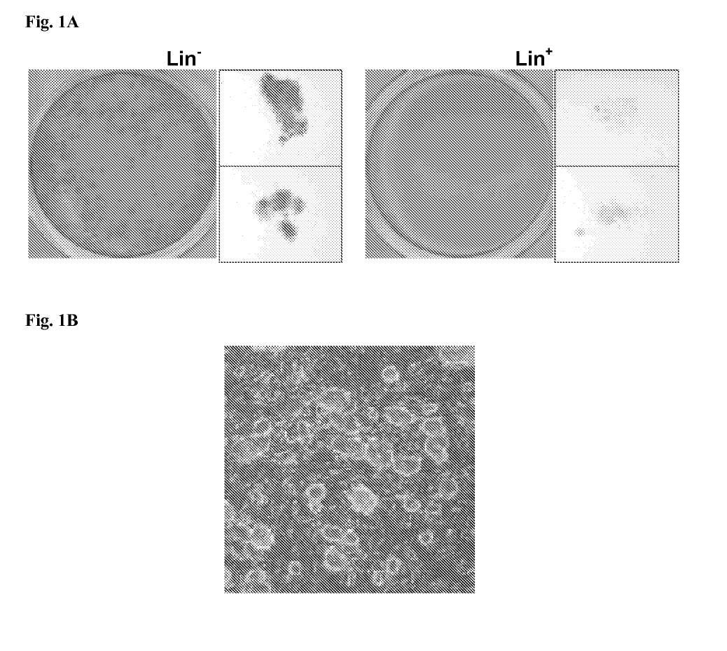

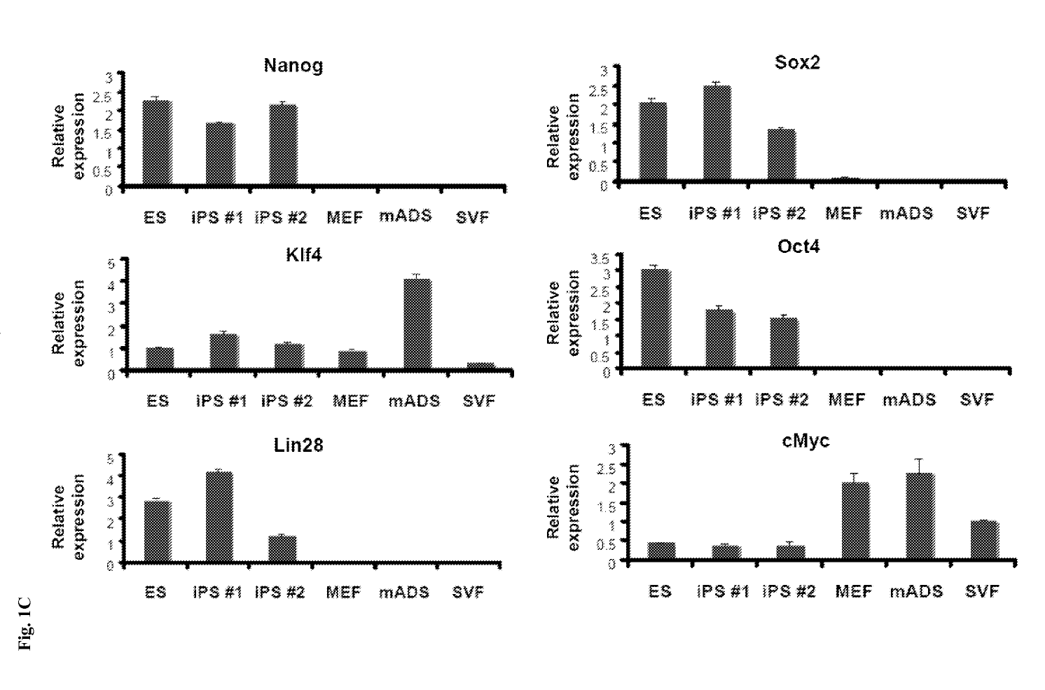

A stromal vascular fraction (SVF) was isolated from white adipose tissue of C57BL/6J mice, and proliferating mouse adipose-derived stem (mADS) cells were enriched by serial plate passaging ^{(8,9)}. mADS cells were retrovirally transduced with c-Myc, Klf4, Oct4 and Sox2, and after 2 days transferred onto plates with or without feeder cell layers from mouse embryonic fibroblasts (MEFs). Interestingly, both conditions resulted in development of Nanog-expressing, ES-like iPS cell colonies at comparable efficiencies (0.25%±0.11% with feeders vs. 0.42%±0.17% without feeders; n=2-3) within 7-10 days, indicating that mADS cells do not require exogenous factors to support the growth of iPS cells. To track down which cell types become iPS cells, freshly isolated SVFs were further separated by lineage cell markers: Lin⁺ for erythrocytes (Ter119), endothelial (CD31) and hematopoietic (CD45) cells, and Lin⁻ for the remaining cells that primarily consist of preadipocytes and mADS cells⁽¹⁰⁾. The results indicate that iPS cells were efficiently generated from Lin⁻ cells, whereas virtually no iPS cells emerged from Lin⁺ cells (FIG. 1A). Among the suggested MSC markers, both mADS and Lin⁻ cells exhibited CD29⁺ (>98%), Sca-1⁺ (40-65%), CD90⁺ (30-55%), CD105⁺ (25-55%), while they were negative for CD34 (>95%), a suggested marker for preadipocytes (FIG. 7A) ^{(10,11)}. Further enrichment of Sca-1⁺, CD90⁺ and CD105⁺ populations in these cells (FIGS. 7A-7B) did not significantly improve the efficiency of iPS cell production (0.21%, 0.29% and 0.33%, respectively). Likewise, SVF Lin⁻ and mADS cells exhibited comparable iPS productivity, suggesting that SVF Lin⁻ and mADS cells are similar cellular populations.

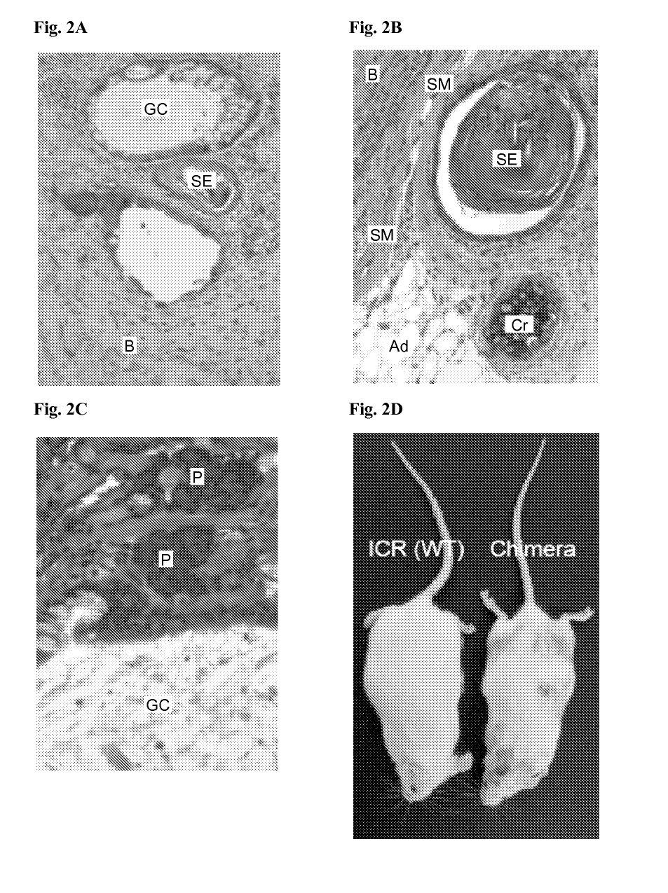

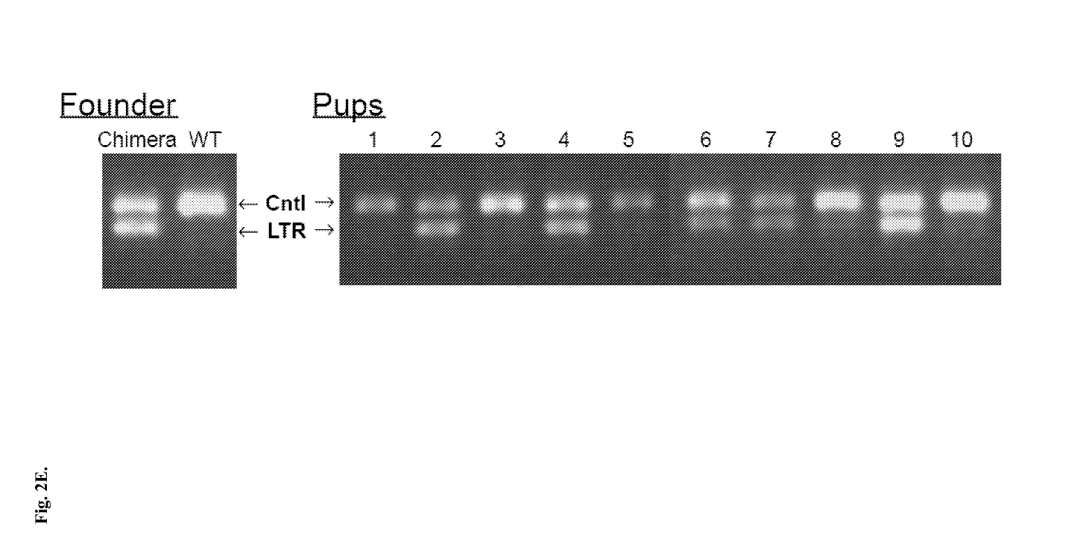

mADS cell-derived iPS clones were expanded (FIG. 1B), and characterized for pluripotency. Quantitative PCR (qPCR) analysis indicated that two independent iPS clones exhibit induction of pluripotent genes, Nanog, Lin28, Sox2 and Oct4, with levels comparable to mouse ES cells (FIG. 1C). Teratomas formed by injecting mADS-derived iPS colonies in vivo showed contributions to all three embryonic germ layers: ectoderm, mesoderm and endoderm (FIG. 2 A-C). Both feeder-grown and feeder-independent adipose iPS cells gave rise to all germ layers at similar levels, indicating their pluripotency. Two iPS clones (on C57BL/6J background with black coat color) were injected into blastocysts from an ICR background (white coat color) for creation of chimeric mice. Successful generation of chimeric mice was achieved (4 chimeras out of 19 with contribution ranging from 5% to 40% from clone #1; 1 chimera out of 17 with 40% contribution from clone #2) as noted by mixed coat color (FIG. 2D). Mating of each of the two chimeras to ICR wild-type mice resulted in germline transmission as revealed by the presence of retroviral construct-specific elements in their offspring (FIG. 2E). Collectively, these results identify mADS cells as a new progenitor depot for iPS cells with in vivo functionality similar to ES cells.

In order to investigate if iPS cells can be created from human adipose sources (hiPS), human c-Myc, Klf4, Oct4 and Sox2 were retrovirally introduced into human white pre-adipocytes (hWP) and adipose-derived mesenchymal stem (hADS) cells. Both cell types efficiently gave rise to hiPS colonies with morphologies similar to human ES cells within 24 days (FIG. 3A). Nanog, SSEA4, and alkaline phosphatase staining indicated that 0.74% (±0.12%; n=3) of hADS cells and 0.31% (±0.01%; n=2) of hWP formed iPS colonies (FIG. 8), compared to 0.28% (±0.08%; n=2) for human keratinocytes, which currently is the most efficient human adult cells to give rise to hiPS cells ⁽¹²⁾. These adipose-derived hiPS clones were then derived and maintained in a feeder-independent, chemically defined medium ^{(13,14)}. By gene expression analysis, different hiPS clones from both hWP and hADS cells exhibited pluripotent markers at levels similar to human ES cells (FIG. 3B). Once hiPS clones were derived, we observed that retroviral-originated transgenes (Oct4, Sox2, c-Myc, and Klf4) were strongly silenced and replaced by induction of endogenous genes in all the clones examined (FIG. 9). An indication of stable reprogramming of somatic cells into pluripotent cells is the robust demethylation of CpG dinucleotides within certain promoter regions of pluripotency associated genes. We employed bisulfite mutagenesis based DNA analysis and found that three independent hiPS clones are hypomethylated at the promoter DMR (differentially methylated region) of the pluripotent gene Oct4 (FIG. 3C). This is in contrast to the original somatic cells, in which their promoter DMR remains highly methylated (FIG. 3C). Collectively, these data demonstrate successful reprogramming of human adipose-derived hiPS cells at the genetic and epigenetic levels. Surprisingly, as observed in murine cells, hiPS colonies also arose from hADS cells in a completely feeder cell-free condition, albeit at lower efficiency (0.008%; n=4). The feeder-free hiPS cells have indistinguishable morphological and proliferation characteristics to hES cells (FIG. 4A), and show pluripotent markers SSEA4 (FIG. 4B), Oct4 and Tra-1-60 (FIG. 4C), and Sox2 (FIG. 4D) as revealed by immunofluorescence. qPCR analysis indicated that feeder-independent hiPS cells express comparable levels of pluripotent markers including Nanog, Oct4, Sox2, Lin28, Zfp42 and Dppa2 (FIG. 4E). The hiPS cells from hWP and feeder-free hADS cells indicated normal karyotypes after extended passages, showing maintenance of chromosomal stability (FIG. 10). This suggests that hADS-derived cells are capable of becoming pluripotent cells independent of feeder layer-originated self-renewal factors.

Recent work demonstrated that human fibroblast-derived hiPS cells can be generated under xenobiotic-free (XF) conditions ⁽¹⁵⁾. For feeder-independent production of hADS-derived hiPS cells, we also avoided exposure of adipose-derived hiPS cells to animal products, by deriving these cells in a defined medium that consists of recombinant protein sources and purified human material ⁽¹⁴⁾. hADS cells were cultured either in XF-MSC serum-free medium or in media containing 2% human serum. The cells were then transduced with virus that had been produced either in medium containing XF Knockout Serum Replacement (XF-KSR) or in xeno- and feeder-free supporting medium (NutriStem), and maintained in NutriStem. Xeno- and feeder-free hiPS colonies were successfully obtained with similar efficiencies (0.007%; n=4). Based on our studies, it is possible to establish an animal source-free, GMP-compliant system by producing adipose-derived hiPS cells in complete xeno- and feeder-free conditions.

In order to test the pluripotency of these hiPS cells, embryoid bodies (EBs) were formed in vitro. The EBs indicated spontaneous induction of differentiation markers from all three genii layers, whereas pluripotent markers were significantly downregulated upon EB formation. See FIGS. 11A-11D. When the EBs were grown in culture plates for 10 days, they exhibited specific proteins for three layers including ectoderm markers GFAP and Tuj (FIG. 5 A and B), mesoderm marker SMA (FIG. 5C), and endoderm marker AFP (FIG. 5D). The in vivo differentiation capability of the hiPS cells was also tested by injecting them subcutaneously into immunodeficient NOD SCID mice. hiPS cells derived from feeder-free hADS (FIG. 5 E and F), hADS with feeders (FIG. 5G), and hWP (FIG. 5H) all formed teratomas and contributed to structures from all three germ layers. Therefore, these results provide in vitro and in vivo functional proof of pluripotency for the adipose-derived hiPS cells.

The discovery that ADS cells do not rely on a feeder layer to become iPS cells prompted us to further explore the mechanism of their feeder independence. Although mechanisms of feeder cells in sustaining pluripotency of ES or iPS cells have not been clearly defined, several secreting factors suggested to be critical for maintenance of self-renewal include leukemia inhibitory factor (LIF) for mouse pluripotent cells and basic FGF (also known as FGF2) for human cells ⁽¹⁶⁾. While MEFs have been routinely used as supporting feeder layers, human foreskin fibroblasts (HFF) were recently developed as xeno-free feeders ⁽¹⁵⁾. We found that mouse ADS cells possess high endogenous expression of factors implicated in self-renewal such as FGF2, TGFβ1, fibronectin-1, vitronectin, activin A and LIF that are relatively comparable to MEFs and higher than other cell types in most cases (FIG. 6A). Human ADS cells also express higher levels of FGF2, TGFβ1, fibronectin-1, vitronectin, and activin A than most other cell lines including HFF (FIG. 6B). In order to directly prove that ADS cells secrete factors to support pluripotency, potential feeder cell lines including hADS, HFF, MEF and mADS cells were mitotically inactivated by gamma irradiation. hADS-derived hiPS cells were then cultured and maintained either by conditioned media taken from irradiated lines or by co-culturing with these lines. Flow cytometry analyses indicate that both hADS- or mADS-conditioned and co-cultured hiPS colonies exhibit comparable pluripotent cell surface markers SSEA3, SSEA4 and Tra-1-60 to those grown with MEF (FIG. 6 C and D). Heterologous hiPS lines (hWP- and hKeratinocyte-derived) were also cultured for extended passages on irradiated lines as feeder layers. The hiPS cells grown on hADS or mADS also showed comparable expression of pluripotent markers to those on MEF (FIG. 12). Taken together, our results suggest remarkable intrinsic capacities of adult adipose-derived cells to support proliferation and maintenance of self-renewal of both autologous and heterologous pluripotent cells.

In conclusion, we demonstrate that adipose-derived cells are easily prepared, virtually unlimited, and a highly capable source of pluripotent cells. Adipose cells join the group of human cell types reported to be amenable to reprogramming, such as fibroblasts, peripheral blood cells, neural stem cells and keratinocytes ^{(12,17,18)}. During the preparation of this manuscript, another group also reported feeder-free derivation of human adipose-derived stem cells ⁽¹⁹⁾. Together with our finding, adipose-derived stem cells are the first human cell type that can achieve induced pluripotency in the complete absence of co-cultured feeder cells. It has yet to be tested if other cell types are also capable of reprogramming in the feeder-free condition. We further explored the mechanism of self-renewal support from adipose-derived stem cells and found that these cells intrinsically express high levels of pluripotency-sustaining factors including basic FGF and LIF. The adipose cells are capable of supporting proliferation and self-renewal of autologous and heterologous hiPS cells as feeder cells, explaining their feeder layer independence to induced pluripotency. Creation of iPS lines from adipose stem cells may be advantageous in providing platforms for treatment of disease models of organs and tissues. In addition, our results offer important clinical and therapeutic implications. For example, an important future question made possible by this work is whether these cells can be reprogrammed toward a brown adipose tissue (BAT) phenotype. Alternatively, as was recently reported for creation of brown adipocytes from fibroblasts ⁽²⁰⁾ and pancreatic beta cells from exocrine cells ⁽²¹⁾, it may be possible to reprogram white adipose-derived ADS cells into brown adipocytes by introducing defined factors. In addition, it was proposed that banking of iPS cell lines may be an ideal option for avoiding immunological rejection during cell transplantation because unlike hES cells, hiPS cells can be derived from patients with matched human leukocyte antigen (HLA) haplotypes ⁽²²⁾. The abundant availability of fat biopsies would make it relatively easy to establish an adipose-derived “iPS library” from individuals with comprehensive HLA haplotypes.

### Materials and Methods

### Cell Isolation

The stromal vascular fraction (SVF) was isolated from subcutaneous and epididymal/parametrial fat pads of 12-week old mice by digestion at 37° C. for 1 h with 1 mg/ml type I collagenase (Worthington) in Hank's buffered salt solution containing 1% BSA, 200 nM adenosine and 50 mg/ml glucose. After sequential filtration through 250 mm and 100 mm nylon filters and centrifugation for 1 min at 400 g, floating adipocytes were removed and washed three times. The pellet (SVF) was treated with erythrocyte lysis buffer (154 mM NH₄Cl, 20 mM Tris pH7.5) and cultured in DMEM containing 10% endotoxin-reduced, heat inactivated FBS (hiFBS; HyClone). SVF was further sorted by IMag streptavidin particles (BD Biosciences), coupled with biotinylated Ter119 (eBioscience), CD31 (BD) and CD45 (BD) mouse-specific antibodies. For mADS cell preparation, erythrocyte-free SVF was plated on bacterial Petri dishes for 1 h to allow hematopoietic cells, including monocytes/macrophages, attach to the dishes. Non-adherent cells were then transferred and cultured in DMEM plus 10% hiFBS, and passaged twice before use for iPS applications. Two independent populations of human ADS cells (hMSC-AT; PromoCell and ADSC; Invitrogen) are derived from subcutaneous fat of a 63-year old Caucasian female and of a 22-year old female, respectively. They were confirmed to be >95% CD44⁺ and >95% CD31⁻/CD45⁻. Similar results of iPS efficiencies and feeder independence were obtained with both of the hADS cells. hWP (PromoCell) is derived from the subcutaneous fat of a 38-year old Caucasian female. Cells were cultured in MesenPRO RS medium (for hADS) or DMEM plus 10% hiFBS (for hWP), and used within four passages. As a control, neonatal human epidermal keratinocytes were obtained from Lonza, and cultured in KGM-2 (Keratinocyte Growth Medium).

### Retrovirus Production and iPS Cell Establishment

Mouse iPS cells were created as previously described, with modifications ^{(23,24)}. Briefly, pMX-based retroviral vectors harbouring each of the mouse reprogramming genes (c-Myc, Klf4, Oct4 or Sox2; Addgene) were transfected along with gag/pol and VSV-G envelope genes into HEK293T cells using Lipofectamine (Invitrogen). Two days after transfection, the supernatant containing viruses was collected and filtered through a 0.45 mm filter. 5×10⁴ each of mADS or SVF cells (passage 2-4) were infected with retrovirus cocktails in 6-well plates (day 0). One well was used to count cell numbers for each group. As controls, cells were transduced with GFP retrovirus alone to test infection efficiencies. On day 2, one fifth of the cells were passed onto gelatin-coated plates with or without MEF feeder layers (Millipore), cultured in Knockout DMEM containing L-glutamine (2 mM), nucleosides (IX), NEAA (1X), b-mercaptoethanol (1%) and LIF (1,000 U/ml), with 15%

KSR (Millipore or Invitrogen). Medium was changed every other day. On days 7 to 10, cells were either immunostained for assessing efficiencies or derived into individual colonies for downstream analyses. Reprogramming of human adipose cells was carried out essentially as described ^{(4,12)}. hWP (5,000 per cm²) or hADS cells (3,000 per cm²) were plated in 6-well plates. The cells were infected with the combination of human reprogramming retroviruses (c-Myc, K1f4, Oct4 or Sox2 in pMXs; Addgene) that had been produced in 293T cells co-transfected with gag/pol and VSV-G as described above. EGFP retrovirus was included at 1/40 volume as internal controls for transduction efficiencies. One well from each group was saved for counting cell numbers. On day 5, cells were passed onto 10-cm dishes covered with feeder MEFs or onto 6-cm dishes without MEFs. Cells were cultured in DMEM/F12 plus 20% KSR supplemented with b-mercaptoethanol (0.1%), NEAA (IX), Glutamax (1%) and 10 ng/ml FGF2 (‘cDF12’ media). Medium was changed every day. On days 18 to 28, individual colonies were picked and cultured feeder-free in defined mTeSR1 medium on plates coated with Matrigel, which was prepared according to the previously described formula ^{(13,14)}, and changed daily. Dispase was used to passage cells. For feeder- and xeno-free (XF) induction of hiPS cells, hADS cells were plated either in StemPro XF-MSC SFM with CELLstart coating (Invitrogen) or in DMEM plus 2% human serum. Retrovirus containing four factors and EGFP was produced in ‘XF-cDF12’ media containing XF-KSR (Invitrogen). 14-21 days after transduction of hADS cells, they were maintained in feeder- and xeno-free medium NutriStem (Stemgent), up to 56 days. Since Matrigel is of mouse tumor origin, all plates for this condition were coated in humanized defined substrate, CELLstart (Invitrogen). Feeder-free production can take longer time periods than feeder-dependent methods; colonies are generally found between 21 and 56 days. For passaging, manual picking was preferred because hiPS cells in the feeder/xeno-free condition were sensitive to digestive enzymes such as dispase and collagenase. All procedures in this study involving hiPS/hES cells was approved by the Embryonic Stem Cell Research Oversight Committee at the Salk Institute.

### Gene Expression Analysis

RNA was extracted from tissues in Trizol (Invitrogen) using a Polytron (Kinematica) and resuspended in DEPC treated water. RNA was DNase (Ambion) treated, reverse transcribed to first-strand cDNA using Superscript II kit (Invitrogen), and then treated with RNAse. Samples were run in triplicate and expression was normalized to the levels of the housekeeping controls, GAPDH for mouse genes or 36b4 for human. Primer sequences are listed in Table 1. Samples were analyzed by qPCR using SYBR Green dye (Invitrogen). qPCR examining endogenous versus exogenous reprogramming genes was performed according to the procedures previously reported ^{(12,23)}. Statistical comparisons in this report were made using Student's t-test. Error bars of the graphs are presented as mean +/−SEM.

### Chimeric Mouse Generation and Genotyping

Mouse iPS cells (on C57BL/6J background) were injected into blastocysts of ICR strains, and implanted into the uterus of 2.5-dpc pseudopregnant mothers to produce chimeric mice. Chimerism was examined after birth by the appearance of black coat color (iPS-derived) from white color background (host). Chimeric mice were bred with wild-type ICR mice to test for germ-line transmission. The presence of iPS-specific components in the chimeric founder and offspring was also investigated by extracting genomic DNA from tail tips and conducting PCR analysis using primers for LTR sequence (specific for pMX constructs) and GAPDH as a control. Primer sequences are found in Table 1.

### In Vitro and in Vivo Differentiation

For in vitro differentiation of mouse iPS cells, cells were trypsinized and cultured in suspension by the hanging-drop method. Cells were then cultured in 10% FBS-containing medium for 7 days to allow spontaneous differentiation before analysis. For differentiation of hiPS cells, embryoid bodies were formed as described previously ^{(12,23)}. For in vivo teratoma formation, 1×106 cells of mouse iPS mixed 1:1 with Matrigel were injected subcutaneously into congenic C57BL/6J strains. For hiPS-derived teratomas, 5-10×106 cells were subcutaneously injected into immunodeficient NOD SCID mice (Jackson Laboratory). After 2-4 weeks (for mouse iPS) or 8-10 weeks (for hiPS), teratomas were dissected, fixed with 10% formalin, prepared for paraffin sections, and stained for H&E. All animal experimental protocols were approved by the Institutional Animal Care and Use Committees at the Salk Institute.

### Promoter Methylation Analysis

Genomic DNA from different hiPS lines and from the corresponding mesenchymal starting populations was extracted from about 1,000,000 cells using QIA AMP DNA Mini Kit (Qiagen). 500 to 900ng of purified DNA was mutagenized with Epigentek (Bionova) according to the manufacturer's specifications. At least 2 different rounds of mutagenesis were carried out for each line analyzed. The promoter sequences of Oct4 were amplified by two subsequent PCR reactions using primers previously described ⁽²⁵⁾. The resulting amplified products were cloned into pGEM T Easy plasmids (Promega), amplified in TOP10F′ cells (Invitrogen), purified and sequenced. Only global C conversions rate higher than 95% were used in the analysis.

### Immunohistochemistry and Cell Staining

Cells grown on dishes were immunostained using the VectaStain ABC kit and ImmPACT DAB substrate (Vector Lab) with rabbit anti-mouse Nanog (Calbiochem), anti-human Nanog (Abcam), or anti-human SSEA4 (Stemgent) antibodies. Alkaline Phosphatase staining was performed using the kit from Stemgent. For immunofluorescent staining, cells grown in 4-well chamber slides were fixed with 4% paraformaldehyde, and incubated with primary antibodies provided in the StemLite Pluripotency Kit (Cell Signaling). Cells were then incubated with secondary antibodies (Alexa Fluor dyes from Invitrogen) and counterstained with Hoechst 33342 for nucleus.

### Flow Cytometry and Cell Sorting

For surface marker analyses, mouse cells were labelled with fluorescence-conjugated anti-mouse antibodies, Alexa647-CD29 (BioLegend), FITC-CD34 (eBioscience), PerCP-Cy5.5-Sca-1 (eBioscience), FITC-CD90.2 (BioLegend), or PE-CD105 (eBioscience), according to the manufacturer's instructions. For feeder supporting analyses of ADS cells, hADS, HFF, MEF and mADS cells were gamma irradiated by cobalt-60. 2×10⁵ (or 1×10⁵ for inserts of coculture plates) cells were then plated each well in 6-well plates. The lines were used in XF-cDF12 media for conditioned media collection, co-culture studies by using Transwell plates (Corning), or feeder layers to support growth of hiPS cells. In conditioned media and co-culture studies, hiPS cells were plated on matrigel.

For flow cytometry analyses of feeder layer cultured hiPS cells, feeder cells were removed by differential gravity after collagenase digestion (i.e. feeder cells in supernatant versus hiPS colonies in pellet) at the last passage, and then hiPS cells were maintained on matrigel with mTeSR1 media for 3 days before the analysis. The cells were trypsinized and analyzed with anti-human antibodies, Alexa647-SSEA3, Alexa488-SSEA4, and Alexa488-Tra-1-60R (BioLegend). Gating was performed with matched isotype control antibodies. DAPI (5 μg/ml) was included in the staining buffer (phenol red-free DMEM plus 2% FBS) to exclude dead cells. Flow cytometry was conducted on a Becton-Dickinson LSR I analyzer. Cells were also sorted at a concentration of 2×10⁷/ml, with the antibodies and DAPI above by using a Becton-Dickinson FACS Vantage SE DiVa.

### III. Tables

TABLE 1
List of primer sequences.

| | | | SEQ | | SEQ |
|---|---|---|---|---|---|
| Gene | | | ID | | ID |
| Name | Species | 5′ end primer | NO: | 3′ end primer | NO: |
| Nanog | m | gcaagcggtggcagaaaaac | 1 | gcaatggatgctgggatactcc | 2 |
| K1f4 | m | atctcaaggcacacctgcgaactcac | 3 | cacacttctggcactgaaaggg | 4 |
| Lin28 | m | gaacatgcagaagcgaagatcc | 5 | gttgatgctttggcaaaagtgg | 6 |
| Oct4 | m | gttggagaaggtggaaccaa | 7 | ctccttctgcagggctttc | 8 |
| Sox2 | m | acagctacgcgcacatga | 9 | ggtagcccagctgctcct | 10 |
| cMyc | m | cctagtgctgcatgaggaga | 11 | tccacagacaccacatcaattt | 12 |
| FGF2 | m | cggctctactgcaagaacg | 13 | tgcttggagttgtagtttgacg | 14 |
| TGFb1 | m | tggagcaacatgtggaactc | 15 | cagcagccggttaccaag | 16 |
| fibro- | m | cggagagagtgcccctacta | 17 | cgatattggtgaatcgcaga | 18 |
| nectin | | | | | |
| vitro- | m | ctggggcagatcctctgat | 19 | actttccctggcacaccat | 20 |
| nectin | | | | | |
| activin A | m | atcatcacctttgccgagtc | 21 | acaggtcactgccttccttg | 22 |
| LIF | m | cccagggatttccaggtact | 23 | tcaactttgccagattccatc | 24 |
| Nanog | h | ccaacatcctgaacctcagc | 25 | gctattcttcggccagttg | 26 |
| K1f4 | h | gggagaagacactgcgtca | 27 | ggaagcactgggggaagt | 28 |
| Lin28 | h | gaagcgcagatcaaaaggag | 29 | gctgatgctctggcagaagt | 30 |
| Oct4 | h | gcaaaacccggaggagtc | 31 | ccacatcggcctgtgtatatc | 32 |
| Sox2 | h | ttgctgcctctttaagactagga | 33 | ctggggctcaaacttctctc | 34 |
| cMyc | h | caccagcagcgactctga | 35 | gatccagactctgaccttttgc | 36 |
| Zfp42 | h | gcgtcataaggggtgagtttt | 37 | agaacattcaagggagcttgc | 38 |
| Dppa2 | h | Tggtgtcaacaactcggtttg | 39 | ctcgaacatcgctgtaatctgg | 40 |
| GATA2 | h | aaggctcgttcctgttcaga | 41 | ggcattgcacaggtagtgg | 42 |
| Nestin | h | tgcgggctactgaaaagttc | 43 | tgtaggccctgtttctcctg | 44 |
| SMA | h | ctgttccagccatccttcat | 45 | tcatgatgctgttgtaggtggt | 46 |
| Actinin | h | gacgtggcagagaagtacctg | 47 | ggcagttccaacgatgtctt | 48 |
| AFP | h | aagaatttcagcatgattttcca | 49 | cacccacttcatggttgcta | 50 |
| Sox7 | h | gagcagtgtggacacgtacc | 51 | gtccaggggagacatttcag | 52 |
| PDX1 | h | aagctcacgcgtggaaag | 53 | gccgtgagatgtacttgttgaa | 54 |
| FGF2 | h | ttcttcctgcgcatccac | 55 | tgcttgaagttgtagcttgatgt | 56 |
| TGFb1 | h | gcagcacgtggagctgta | 57 | cagccggttgctgaggta | 58 |
| fibro- | h | ctggccgaaaatacattgtaaa | 59 | ccacagtcgggtcaggag | 60 |
| nectin | | | | | |
| vitro- | h | tgagtgcaagccccaagt | 61 | gccatcgtcatagaccgtgt | 62 |
| nectin | | | | | |
| activin A | h | ctcggagatcatcacgtttg | 63 | ccttggaaatctcgaagtgc | 64 |
| LTR | ret | ggaatgaaagaccccacctgtag | 65 | gcgagaagcgaactgattggttag | 66 |

Species: m: mouse; h: human; ret: retrovirus construct

### IV. References

1. Fraser J K, et al., 2006, Trends Biotechnol 24(4):150-154.

2. Zuk P A, et al., 2002, Mol Biol Cell 13(12):4279-4295.

3. Park I H, et al., 2008, Nature 451(7175):141-146.

4. Takahashi K, et al., 2007, Cell 131(5):861-872.

5. Takahashi K & Yamanaka S, 2006, Cell 126(4):663-676.

6. Yu J, et al., 2007, Science 318(5858):1917-1920.

7. Martin M J, et al., 2005, Nat Med 11(2):228-232.

8. Bunnell B A, et al., 2008, Methods 45(2):115-120.

9. Mitchell J B, et al., 2006, Stem Cells 24(2):376-385.

10. Rodeheffer M S, et al., 2008, Cell 135(2):240-249.

11. Sengenes C, et al., 2005, J Cell Physiol 205(1):114-122.

12. Aasen T, et al., 2008, Nat Biotechnol 26(11):1276-1284.

13. Ludwig T E, et al., 2006, Nat Methods 3(8):637-646.

14. Ludwig T E, et al., 2006, Nat Biotechnol 24(2):185-187.

15. Rodriguez-Piza I, et al., 2009, Stem Cells. 28(1):36-44.

16. Xu R H, et al., 2005, Nat Methods 2(3):185-190.

17. Kim J B, et al., 2009, Nature 461(7264):649-653.

18. Loh Y H, et al., 2009, Blood 113(22):5476-5479.

19. Sun N, et al., 2009, Proc Natl Acad Sci USA 106(37):15720-15725.

20. Kajimura S, et al., 2009, Nature 460(7259):1154-1158.

21. Zhou Q, et al., 2008, Nature 455(7213):627-632.

22. Nakatsuji N, et al., 2008, Nat Biotechnol 26(7):739-740.

23. Kawamura T, et al., 2009, Nature 460(7259):1140-1144.

24. Takahashi K, et al., 2007, Nat Protoc 2(12):3081-3089.

25. Freberg C T, et al., 2007, Mol Biol Cell 18(5):1543-1553.

1. A method for preparing an adipose-derived induced pluripotent stem cell comprising:
(i) transfecting an adipose-derived stem cell with a nucleic acid encoding a cMYC protein, a nucleic acid encoding an OCT4 protein, a nucleic acid encoding a KLF4 protein and a nucleic acid encoding a SOX2 protein to form a transfected adipose-derived stem cell; and
(ii) allowing said transfected adipose-derived stem cell to divide thereby forming said adipose-derived induced pluripotent stem cell.

2. The method of claim 1, wherein adipose-derived stem cell is a pre-adipocyte stem cell.

3. The method of claim 1, wherein said allowing said transfected adipose-derived stem cell to divide in step (ii) occurs in a xeno-free environment.

4. The method of claim 1, wherein said allowing said transfected adipose-derived stem cell to divide in step (ii) occurs in the absence of feeder cells.

5. The method of claim 1, wherein said adipose-derived stem cell is an adipose-derived mesenchymal stem cell.

6. The method of claim 1, wherein said adipose-derived stem cell is a white pre-adipocyte.

7. An adipose-derived induced pluripotent stem cell prepared in accordance with the method of claim 1.

8. An adipose-derived stem cell comprising a nucleic acid encoding a cMYC protein, a nucleic acid encoding an OCT4 protein, a nucleic acid encoding a KLF4 protein and a nucleic acid encoding a SOX2 protein.

9. A method for producing a somatic cell comprising:
(a) contacting an adipose-derived induced pluripotent stem cell with cellular growth factors; and
(b) allowing said adipose-derived induced pluripotent stem cell to divide, thereby forming said somatic cell.

10. The method of claim 9, wherein said adipose-derived induced pluripotent stem is prepared in accordance with a method comprising:
(i) transfecting an adipose-derived stem cell with a nucleic acid encoding a cMYC protein, a nucleic acid encoding an OCT4 protein, a nucleic acid encoding a KLF4 protein and a nucleic acid encoding a SOX2 protein to form a transfected adipose-derived stem cell; and
(ii) allowing said transfected adipose-derived stem cell to divide thereby forming said adipose-derived induced pluripotent stem cell.

11. The method of claim 10, wherein said allowing said transfected adipose-derived stem cell to divide in step (ii) occurs in the absence of feeder cells.

12. A method of treating a mammal in need of tissue repair comprising:
(i) administering an adipose-derived induced pluripotent stem to said mammal; and
(ii) allowing said adipose-derived induced pluripotent stem cell to divide and differentiate into somatic cells in said mammal, thereby providing tissue repair in said mammal.

13. The method of claim 12, wherein said adipose-derived induced pluripotent stem is prepared in accordance with a method comprising:
(i) transfecting an adipose-derived stem cell with a nucleic acid encoding a cMYC protein, a nucleic acid encoding an OCT4 protein, a nucleic acid encoding a KLF4 protein and a nucleic acid encoding a SOX2 protein to form a transfected adipose-derived stem cell; and
(ii) allowing said transfected adipose-derived stem cell to divide thereby forming said adipose-derived induced pluripotent stem cell.

14. The method of claim 13, wherein said allowing said transfected adipose-derived stem cell to divide in step (ii) occurs in the absence of feeder cells.

15-21. (canceled)
